(19) United States
(12) Patent Application Publication
(10) Pub. No.: US 2021/0369138 A1
(43) Pub. Date: Dec. 2, 2021

(54) SYSTEM AND METHOD FOR DETECTING RESPIRATORY INFORMATION USING CONTACT SENSOR

(71) Applicant: KONINKLIJKE PHILIPS N.V., EINDHOVEN (NL)

(72) Inventors: Salvatore SAPORITO, Rotterdam (NL); Josef Heribert BALDUS, Aachen (NL)

(21) Appl. No.: 17/120,431

(22) Filed: Dec. 14, 2020

Related U.S. Application Data
(60) Provisional application No. 62/949,487, filed on Dec. 18, 2019.

(51) Int. Cl. A61B 5/08; A61B 5/113; A61B 5/00; A61B 5/11

(52) U.S. Cl. CPC A61B 5/0816; A61B 5/113; A61B 5/6844; A61B 5/1116; A61B 5/4809; A61B 5/6802

(57) ABSTRACT

A method for monitoring a patient includes receiving sensor signals from a sensor arrangement, extracting movement information from the sensor signals, determining a sensing period between the sensor arrangement and a body part of a patient based on the movement information, and determining a respiratory rate of the patient based on the sensor signals occurring during the period of contact. The sensor signals may be received from a sensor arrangement incorporated on or within a wearable item that moves relative to the body part of the patient. The sensor arrangement is in intermittent patterns of contact and non-contact with patient as a result of movement of the wearable item. The wearable item may be, for example, a pendant on a necklace.

### CROSS-REFERENCE TO PRIOR APPLICATIONS

This application claims the benefit of U.S. Provisional Application No. 62/949,487, filed on 18 Dec. 2019. This application is hereby incorporated by reference herein.

### TECHNICAL FIELD

This disclosure relates generally to processing information, and more specifically, but not exclusively, to detecting and processing sensor signals indicative of physiological information.

### BACKGROUND

Monitoring respiration may provide important information concerning the health of a patient. Physiologically, the lung movement required to effect respiration is accomplished by movement of the diaphragm and external intercostal muscles. When the diaphragm contracts, a pressure differential is created that causes air to enter the lungs. This action is coordinated with contraction of the intercostal muscles, which contraction causes the ribs to elevate and expand the total chest cavity, thereby allowing a greater volume of air to enter. The inhaled air coupled with expansion of the chest wall cause the diameter of the chest to increase by up to several centimeters in a healthy patient.

In terms of morbidity, respiratory rate is perhaps the most relevant vital sign to monitor in a transitional post-acute frail elderly population, especially during a post-hospital discharge phase. Vast clinical literature suggests that changes in respiratory rate are strong predictors of adverse events, such as cardiac arrest and intensive care admission after visits to the emergency department. The intensive care admission may be caused by, for example, exacerbation of chronic respiratory conditions such as chronic obstructive pulmonary disease. Respiratory rate monitoring may also offer insight on other metabolic-related conditions, such as diabetics ketoacidosis, toxicological reactions, and dehydration induced by thermal stress.

For many patients (and especially those having some of the conditions mentioned above), it would be beneficial to monitor respiratory rate at home or in surroundings outside of a hospital or other medical facility. Two examples of where home-monitoring of respiratory rate may be of particular relevance is in the detection of sleep apnea and sudden respiratory depression in post-surgical patients.

Existing monitors for detecting respiratory rate have a number of drawbacks. For example, existing monitors must be applied to patients by trained professionals in a specialized manner. This requires the patient to go to a hospital or other medical setting, which is inconvenient and full of delays. Also, existing monitors are required to be fixed to the patient in order to obtain accurate readings. This significantly restricts the ability of the patient to perform normal activities, for example, as would be performed at a home or another type of non-clinical setting. The foregoing drawbacks translate into additional problems. For example, the use of fixed monitors in a clinical setting limits the time to measure respiratory rate to a small window, e.g., only during the period when the patient is at the clinical setting and the monitor is actually fixed to him. Consequently, measuring respiratory rate repeatedly throughout the day and night is neither feasible nor practical with existing respiratory rate monitors.

### SUMMARY

A brief summary of various example embodiments is presented below. Some simplifications and omissions may be made in the following summary, which is intended to highlight and introduce some aspects of the various example embodiments, but not to limit the scope of the invention. Detailed descriptions of example embodiments adequate to allow those of ordinary skill in the art to make and use the inventive concepts will follow in later sections.

In accordance with one or more embodiments, a method for monitoring a patient includes receiving sensor signals from a sensor arrangement; extracting movement information from the sensor signals; determining a sensing period between the sensor arrangement and a body part of a patient based on the movement information; and determining a respiratory rate of the patient based on the sensor signals occurring during the sensing period, wherein the sensor signals are received from a sensor arrangement incorporated on or within a wearable item that moves relative to the body part of the patient, the sensor arrangement in intermittent patterns of contact and non-contact with patient as a result of movement of the wearable item. The wearable item may be a pendant on a necklace.

The sensing period may include a period of contact between the sensor arrangement and the body part of the patient. Determining the sensing period may include determining one or more periods of non-contact between the sensor arrangement and the body part of the patient, and excluding the one or more periods of non-contact to determine a period of contact between the sensor arrangement and the body part of the patient, the period of contact corresponding to the sensing period.

The movement information may indicate movement of the wearable item along a subset of three directional axes. The subset may include one axis, and excluding the remaining two axes, of the three directional axes, or a combination of two of the three directional axes. The method may include combining the sensor signals generated along the combination of two of the three directional axes to generate the movement information.

Determining the period of contact may include determining at least one time window where the movement information indicates that movement of the wearable item along the subset of three directional axes is below at least a first predetermined value. The first predetermined value may be indicative of a sitting state, a lying down state, standing still, or a sleep state.

Determining the at least one time window may include identifying a plurality of candidate time windows, ranking the candidate time windows based on at least one parameter, and selecting the at least one time window from the plurality of candidate time windows, wherein the at least one parameter corresponds to at least one parameter of the sensor signals in each of the plurality of candidate time windows and wherein unselected ones of the candidate time windows are discarded as containing noise or spurious signals. The at least one parameter of the sensor signals may be based on amplitudes of the sensor signals in the plurality of candidate windows. The at least one parameter of the sensor signals may be based on sensitivity of the sensor arrangement. The at least one parameter of the sensor signals may be based on a median value of the sensor signals in the plurality of candidate time windows.

The method may include generating median values based on amplitudes of the sensor signals during one or more candidate respiratory intervals corresponding to the sensing period, the median values generated for at least a subset of three directional axes and indicative of one or more corresponding orientations of the wearable item; generating variance values for the sensor signals during the one or more candidate respiratory intervals corresponding to the sensing period, the variance values, the variance values generated for at least the subset of the three directional axes and indicative of one or more corresponding motion levels of the wearable item; and determining the period of contact between the sensor arrangement and the body part of a patient based on one or more of the median values and one or more of the variance values. Determining the respiratory rate may include generating power spectral and cross-spectral estimates based on the sensor signals in the sensing period and calculating the respiratory rate based on the power spectral estimates.

In accordance with one or more other embodiments, a monitor includes a memory configured to store instructions; and a processor configured to execute the instructions to generating information for a patient to be monitored, the processor including: (a) a contact detector configured to receive sensor signals from a sensor arrangement, extract movement information from the sensor signals, and determine a sensing period between the sensor arrangement and a body part of a patient based on the movement information, and (b) a respiratory rate calculator configured to determine a respiratory rate of the patient based on the sensor signals occurring during the sensing period, wherein the sensor signals are received from a sensor arrangement incorporated on or within a wearable item that moves relative to the body part of the patient, the sensor arrangement in intermittent patterns of contact and non-contact with patient as a result of movement of the wearable item.

The sensing period may include a period of contact between the sensor arrangement and the body part of the patient. Determining the sensing period may include determining one or more periods of non-contact between the sensor arrangement and the body part of the patient, and excluding the one or more periods of non-contact to determine a period of contact between the sensor arrangement and the body part of the patient, the period of contact corresponding to the sensing period. The movement information may indicate movement of the wearable item along a subset of three directional axes. The subset may include one axis, and excluding the remaining two axes, of the three directional axes, or a combination of two of the three directional axes.

The contact detector may be configured to combine the sensor signals generated along the combination of two of the three directional axes to generate the movement information. The contact detector may determine the sensing period by determining at least one time window where the movement information indicates that movement of the wearable item along the subset of three directional axes is below at least a first predetermined value. The first predetermined value may be indicative of a sitting state, a lying down state, standing still, or a sleep state.

The contact detector may determine the at least one time window by identifying a plurality of candidate time windows, ranking the candidate time windows based on at least one parameter, and selecting the at least one time window from the plurality of candidate time windows, wherein the at least one parameter corresponds to at least one parameter of the sensor signals in each of the plurality of candidate time windows and wherein unselected ones of the candidate time windows are discarded as containing noise or spurious signals. The at least one parameter of the sensor signals may be based on amplitudes of the sensor signals in the plurality of candidate windows, sensitivity of the sensor arrangement, or both.

The accompanying figures, where like reference numerals refer to identical or functionally similar elements throughout the separate views, are incorporated in and form part of the specification, and serve to illustrate example embodiments of concepts found in the claims and explain various principles and advantages of those embodiments. These and other specific features are more fully disclosed in the following specification, reference being had to the accompanying drawings, in which:

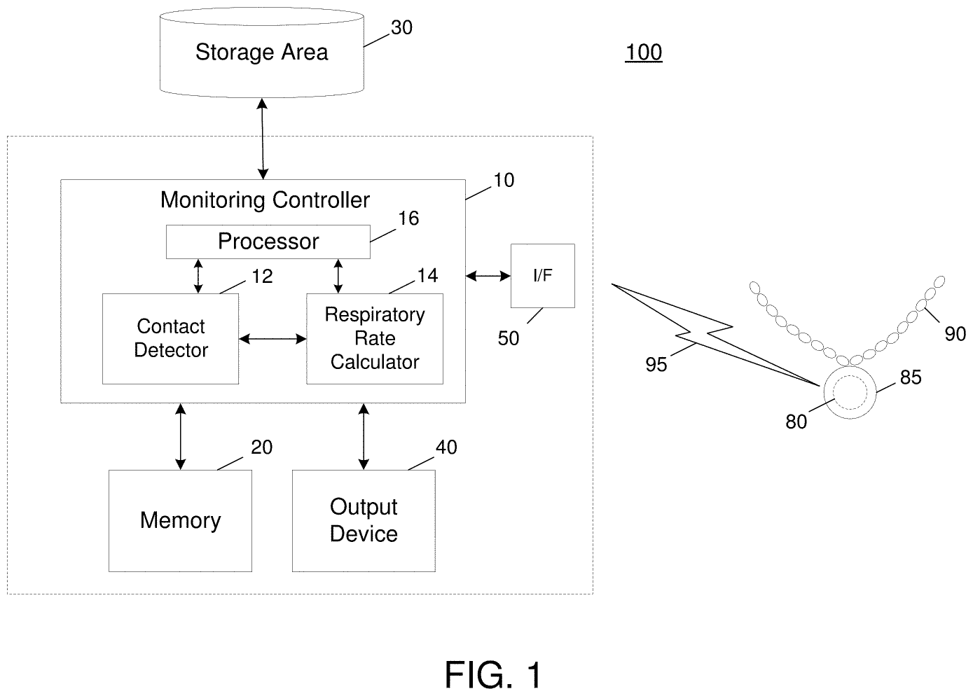

FIG. 1 illustrates an embodiment of a monitoring system (or monitor) for determining respiratory information;

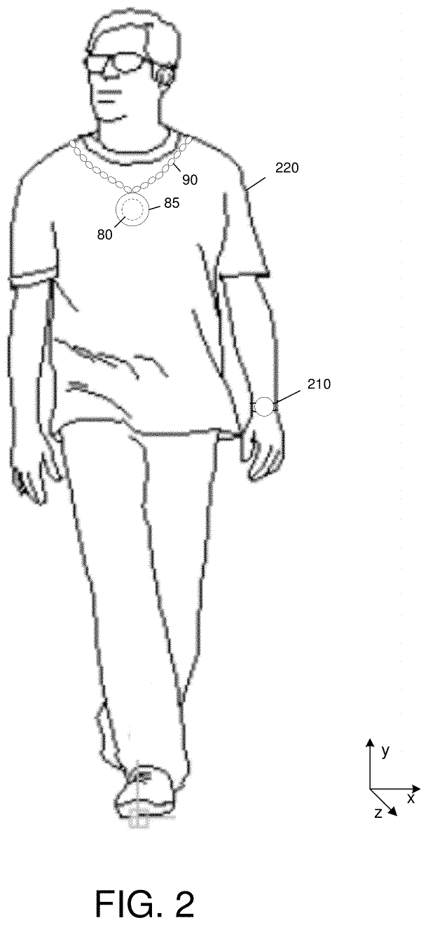

FIG. 2 illustrates an example of a wearable item including a sensor arrangement;

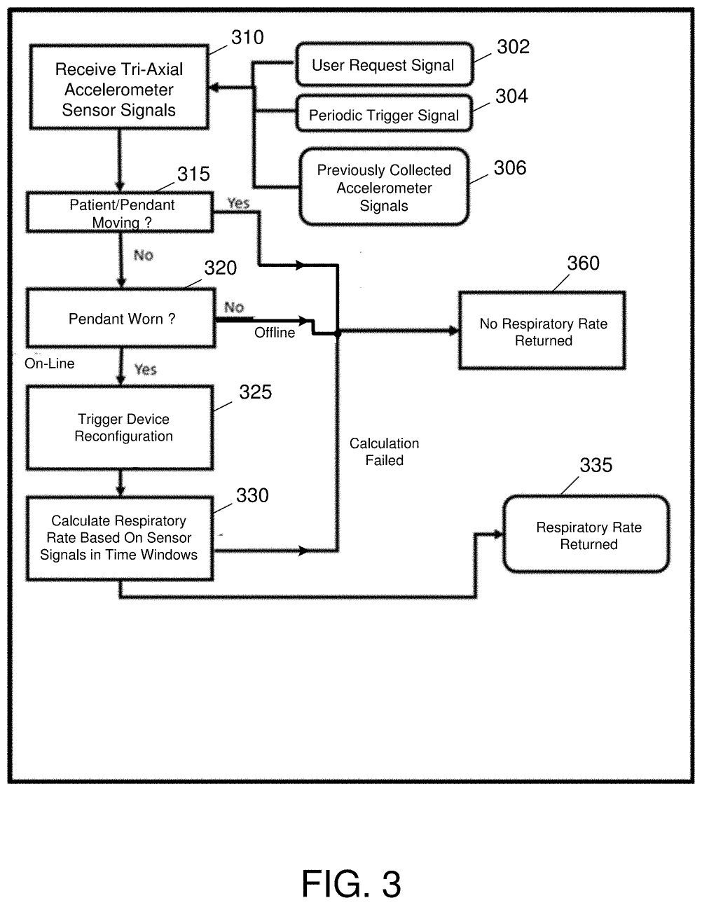

FIG. 3 illustrates an embodiment of a method for determining respiratory information;

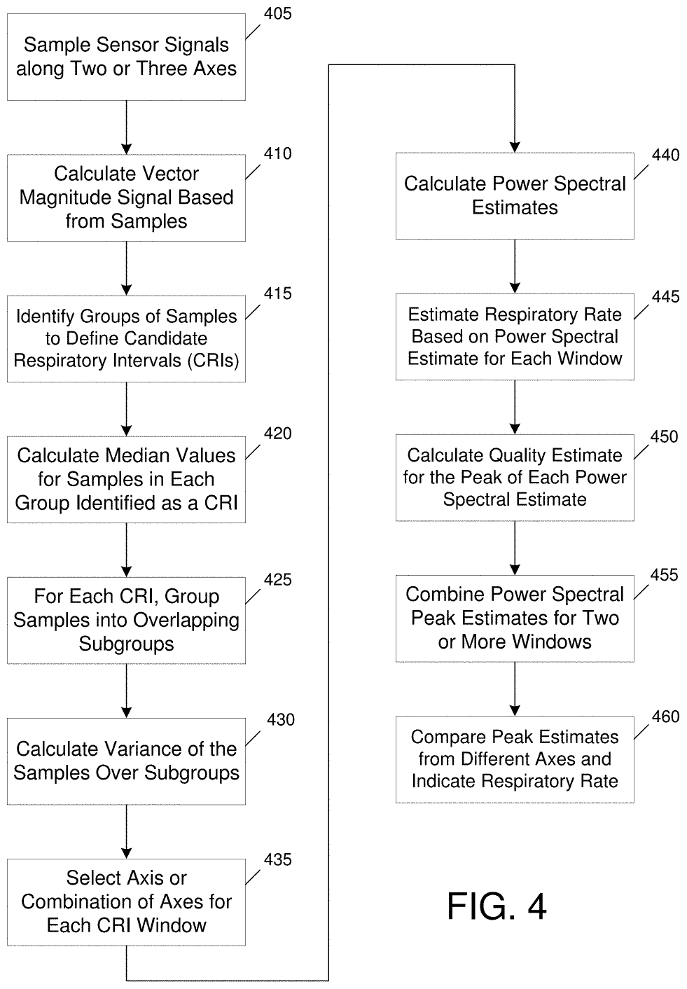

FIG. 4 illustrates an embodiment of a method for calculating respiratory information;

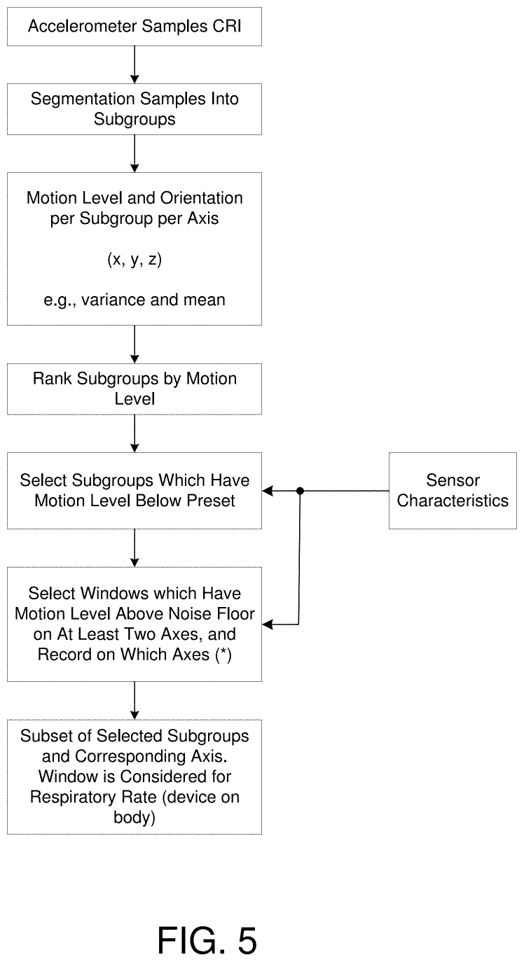

FIG. 5 illustrates an embodiment of a method for determining respiratory information;

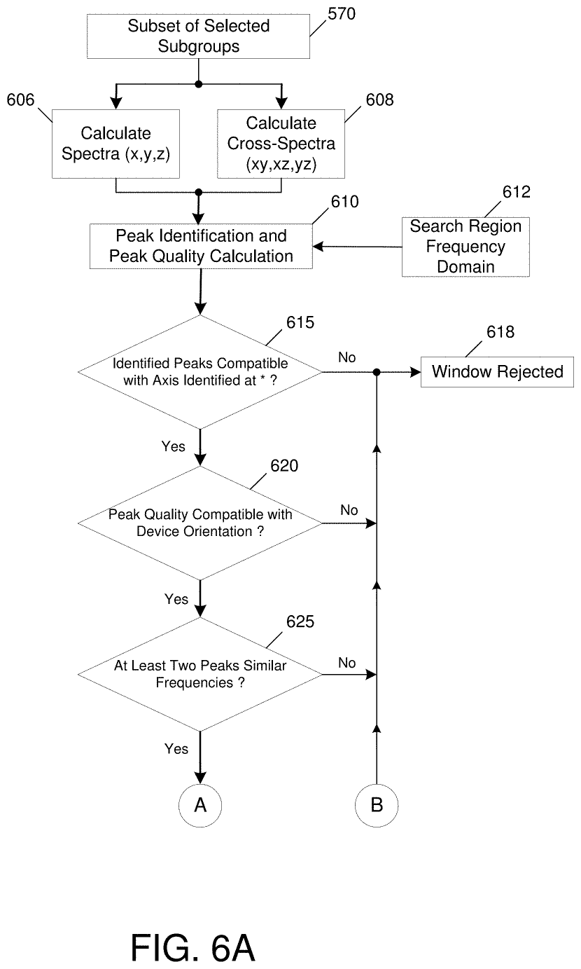

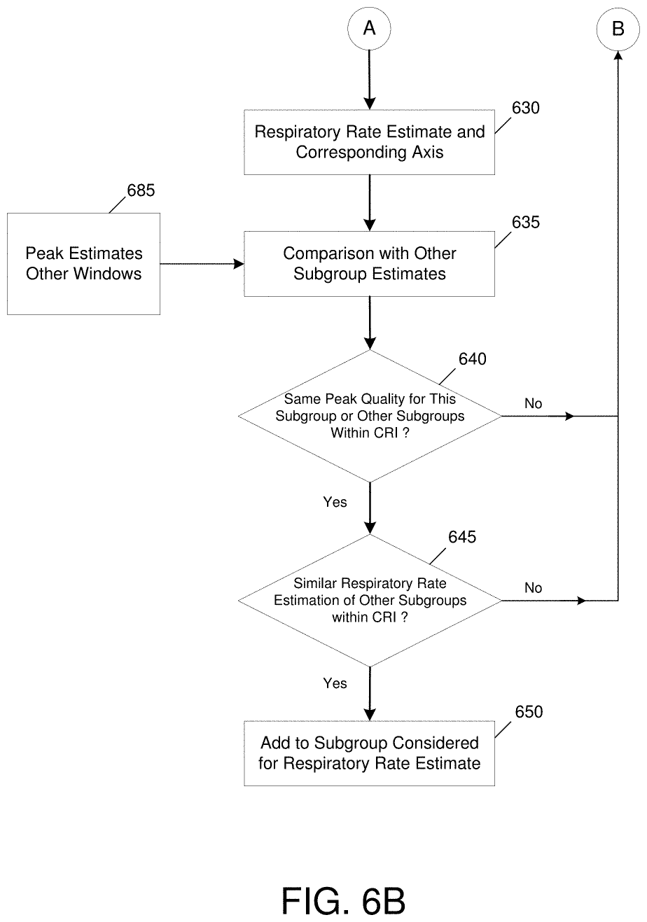

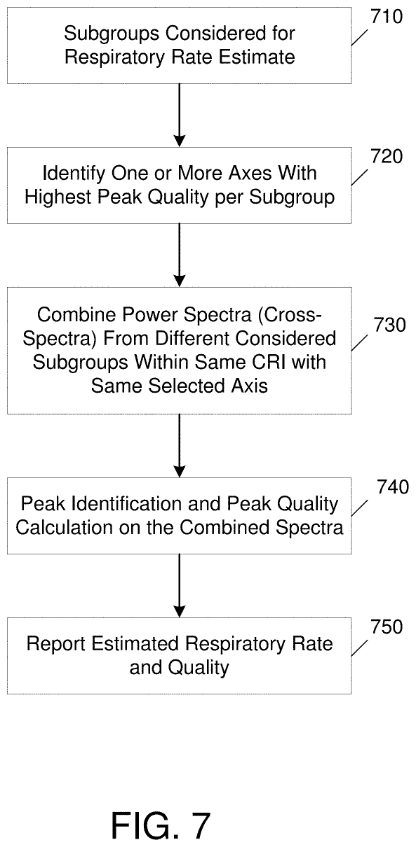

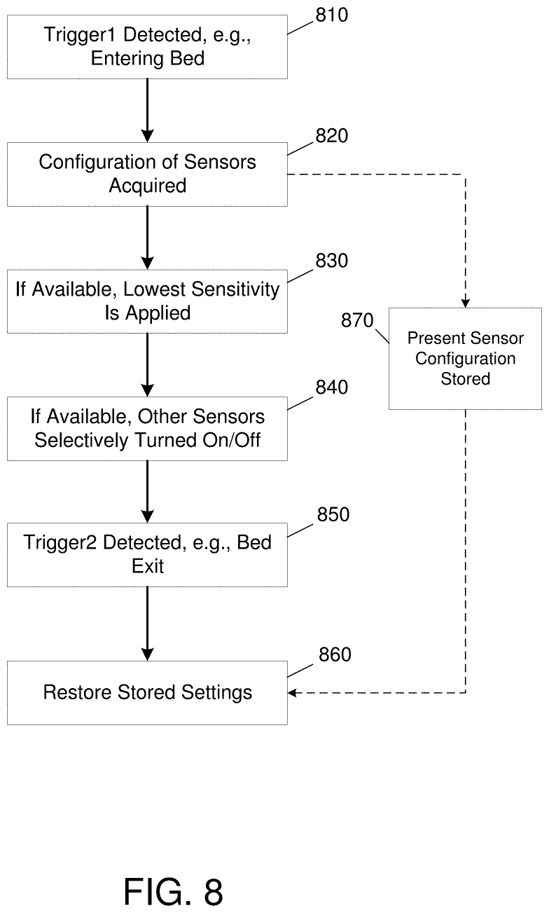

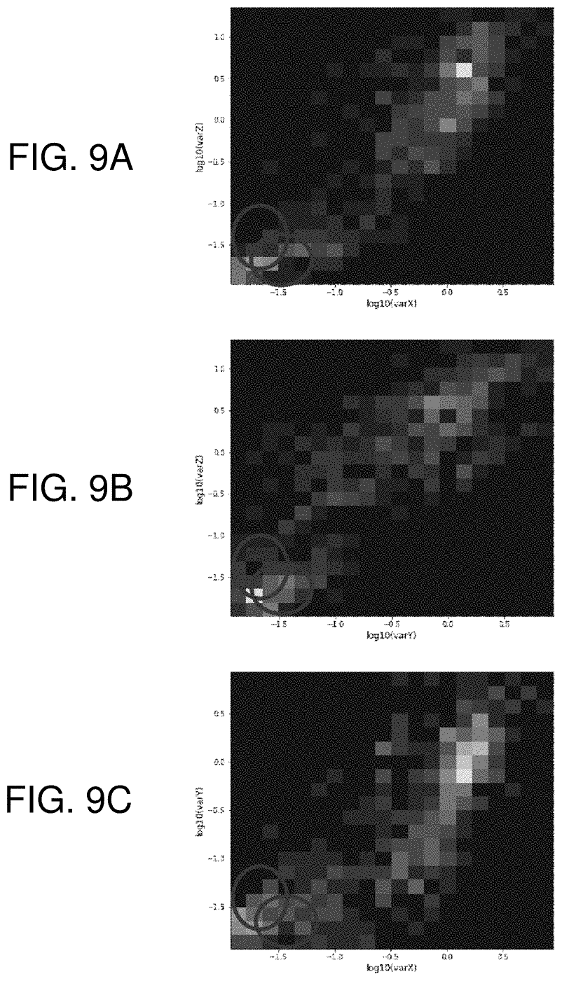

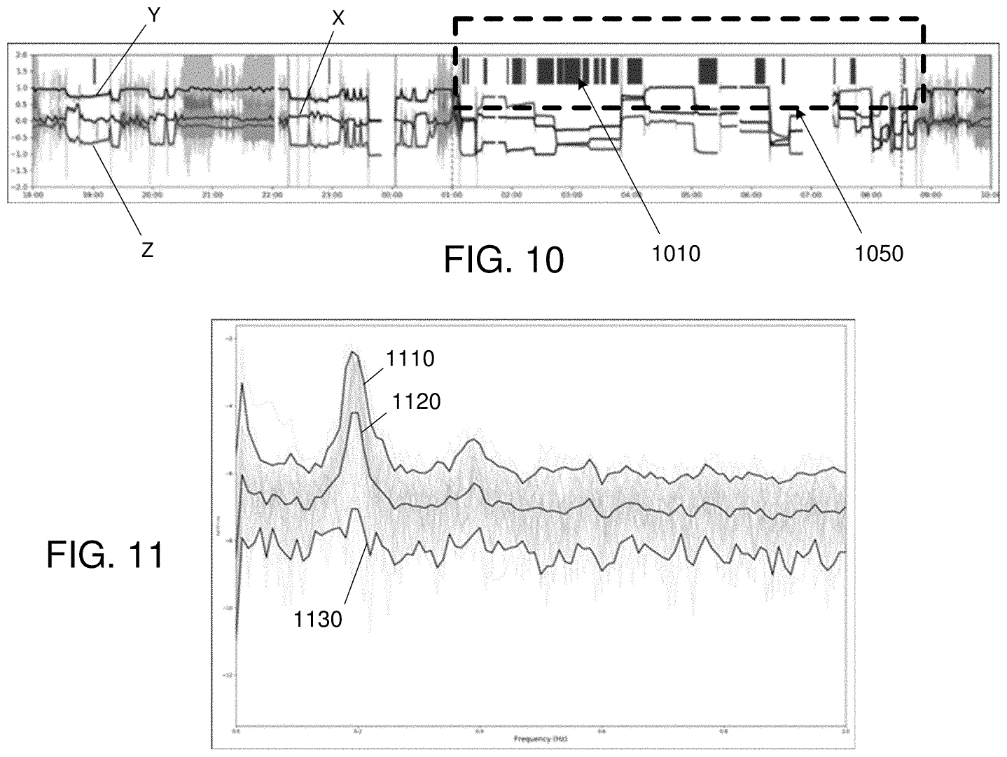

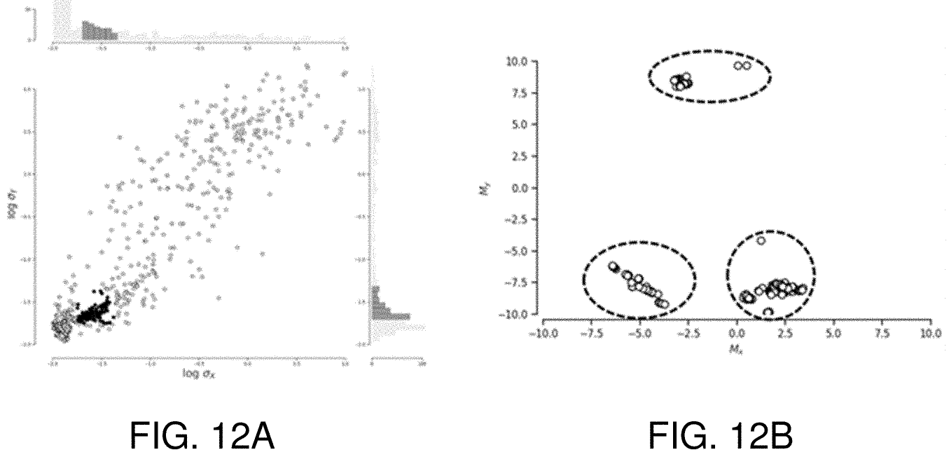

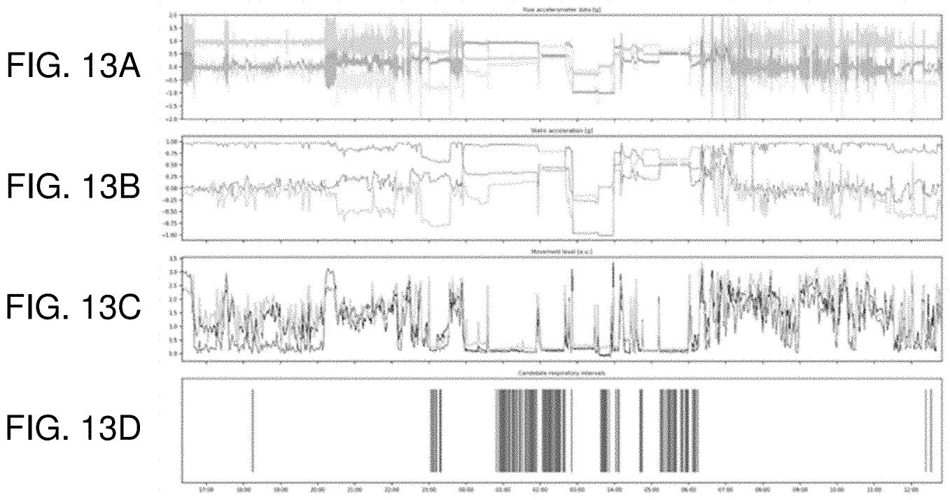

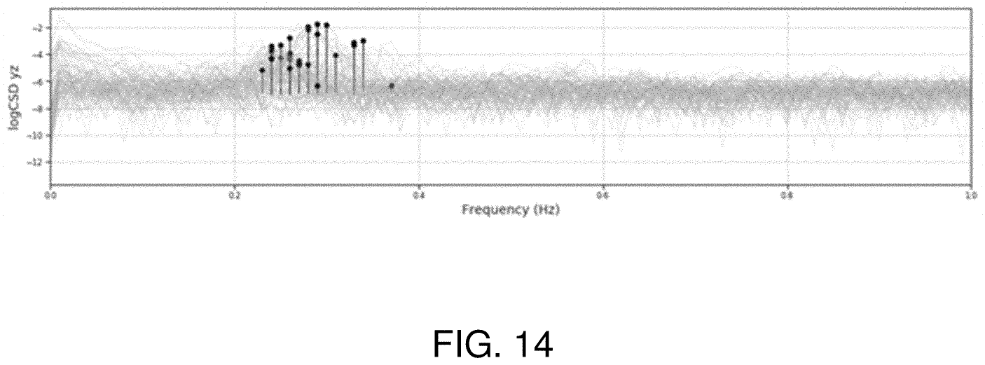

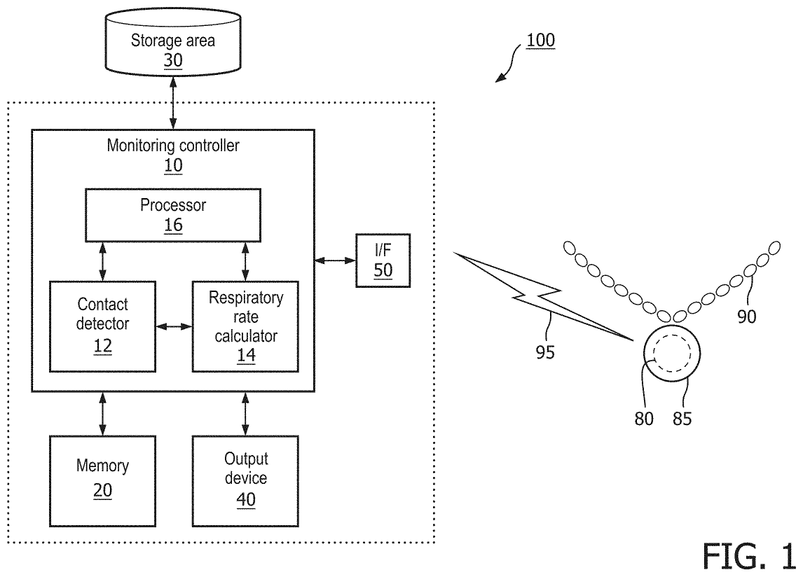

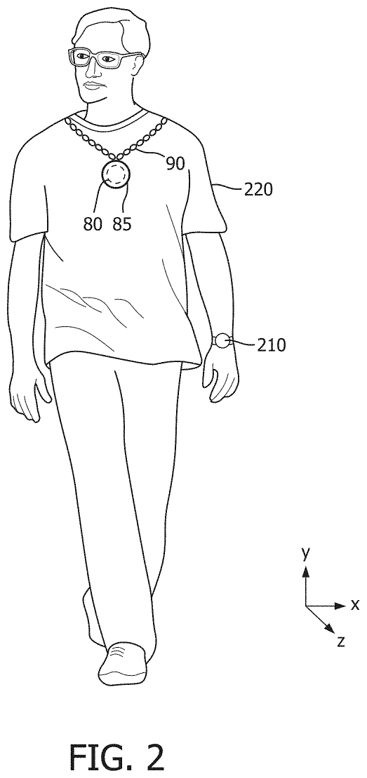

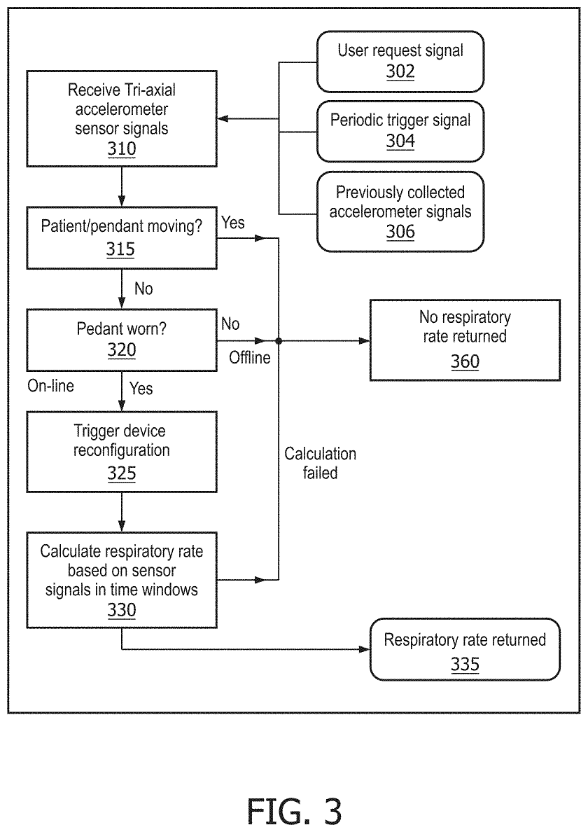

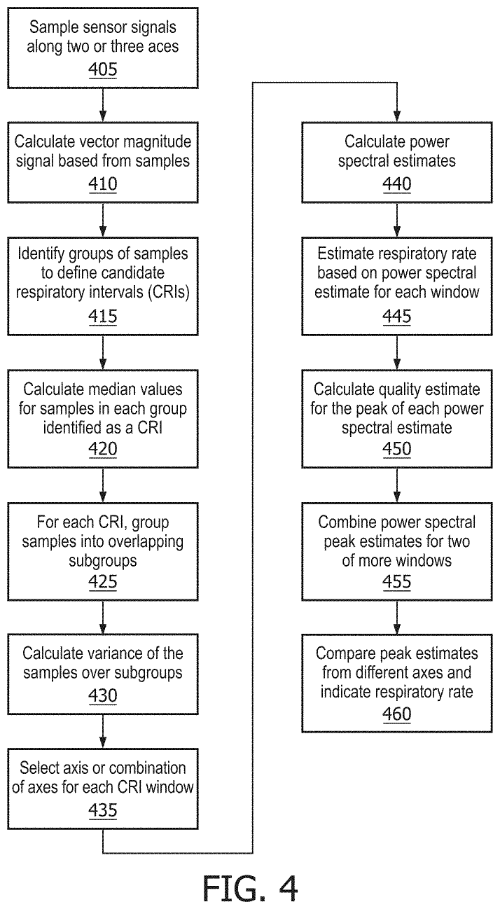

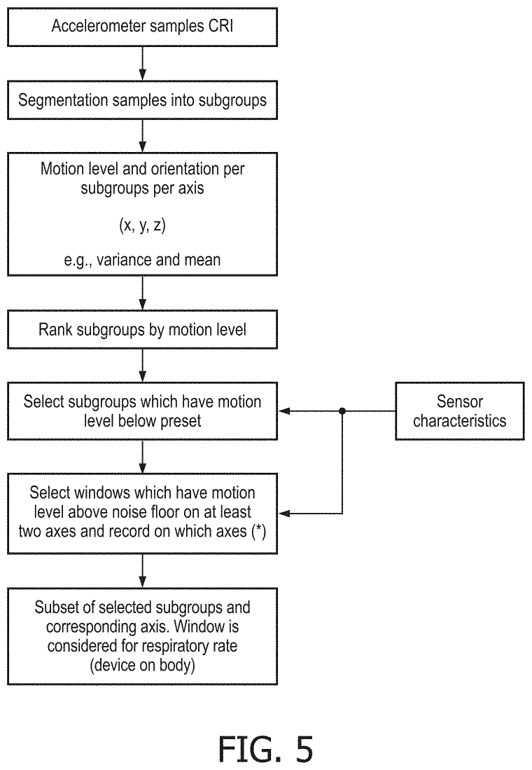

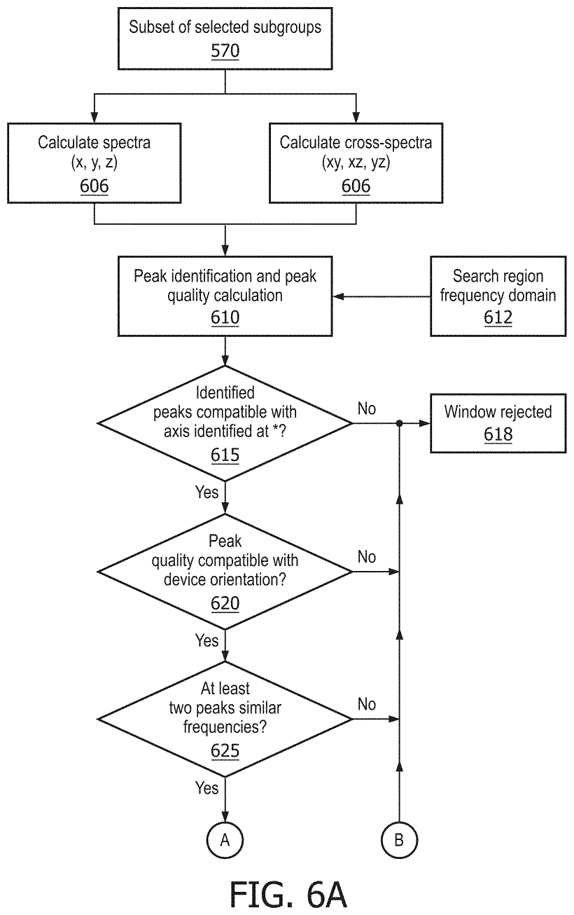

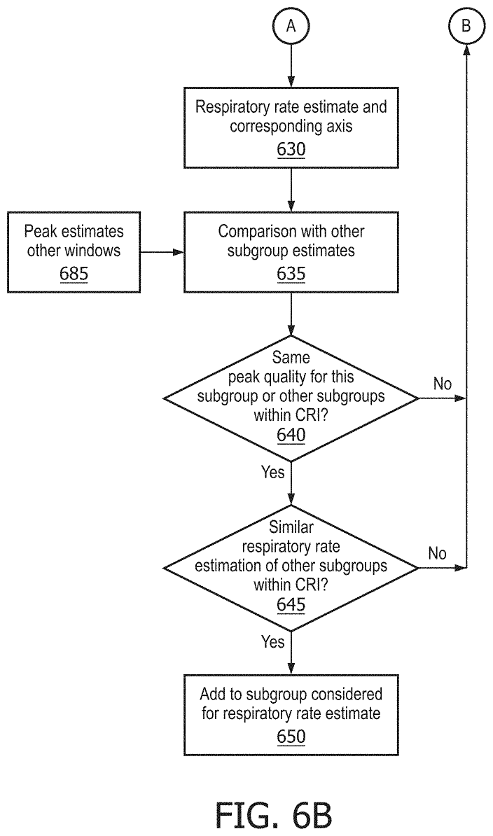

FIGS. 6A and 6B illustrate additional operations of the method of FIG. 5;

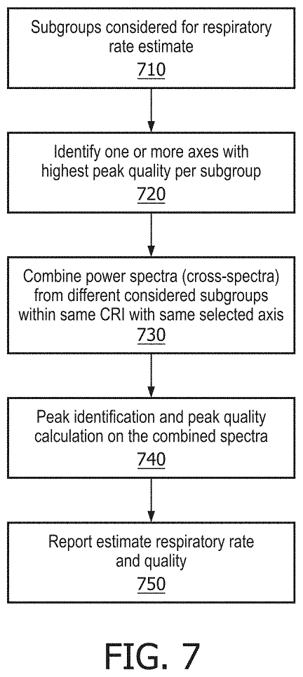

FIG. 7 illustrates additional operations of the methods of FIGS. 5 and 6;

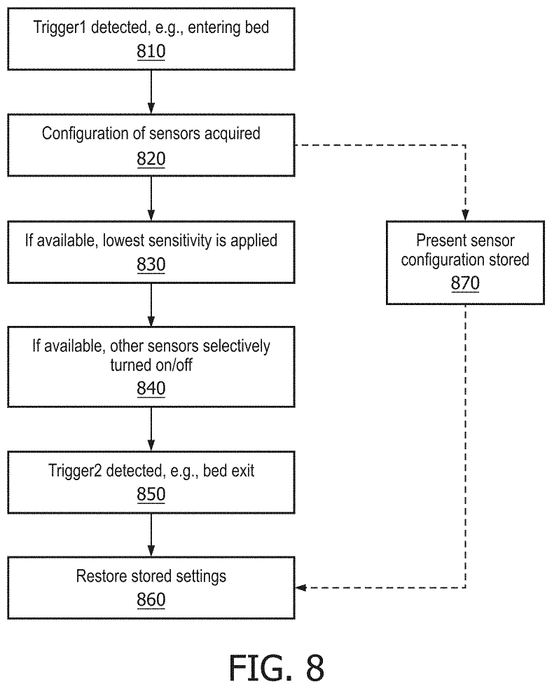

FIG. 8 illustrates an embodiment of a method for reconfiguring a trigger device;

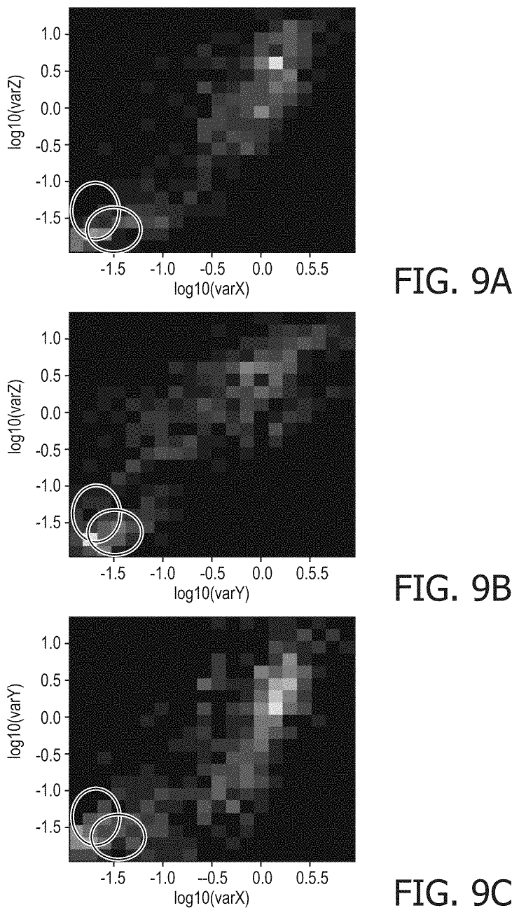

FIGS. 9A to 9C illustrate examples of joint distributions of motion levels between different axes of a sensor arrangement;

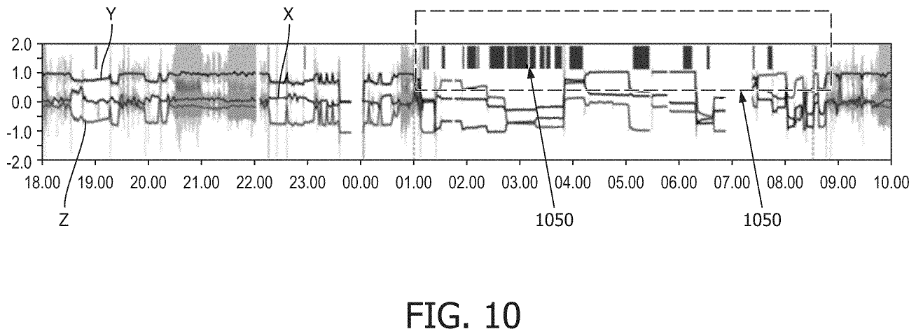

FIG. 10 illustrates an example of a graph of accelerometer signals;

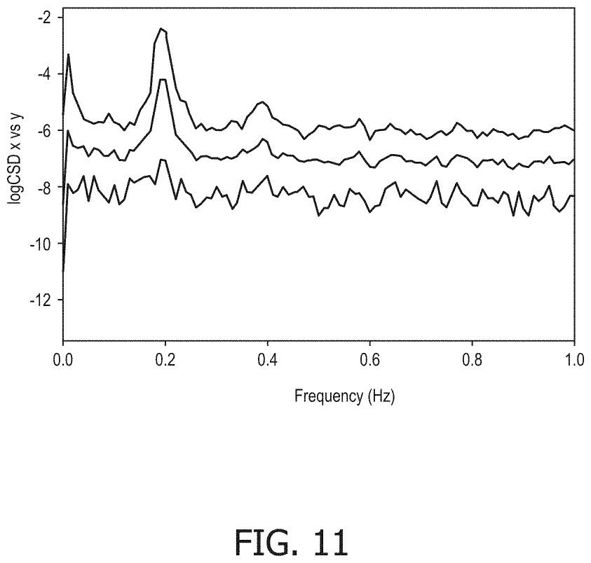

FIG. 11 illustrates a graph including examples of multiple cross-spectral estimates calculated for multiple windows within one or more of the same CRIs;

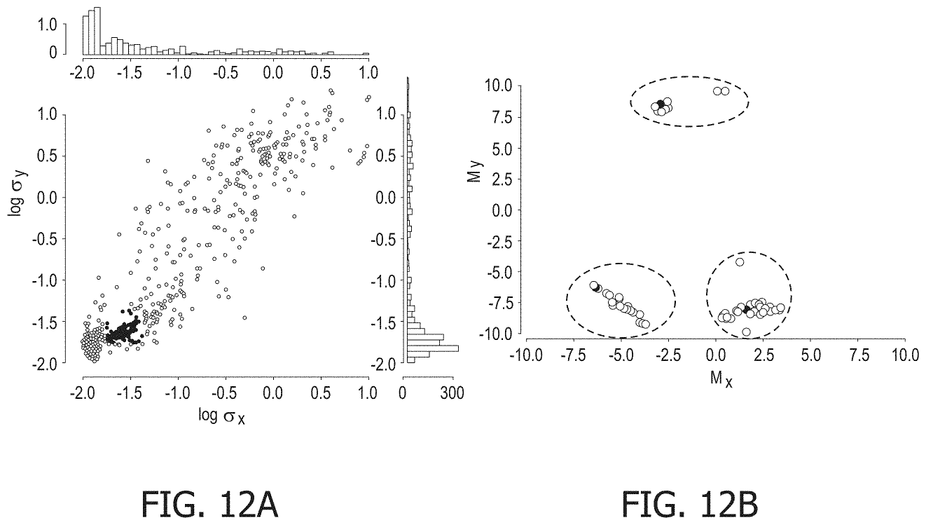

FIG. 12A illustrates examples motion levels for two accelerometer axes, and FIG. 12B illustrates examples of median accelerometer values for two axes to be used for a selection of windows;

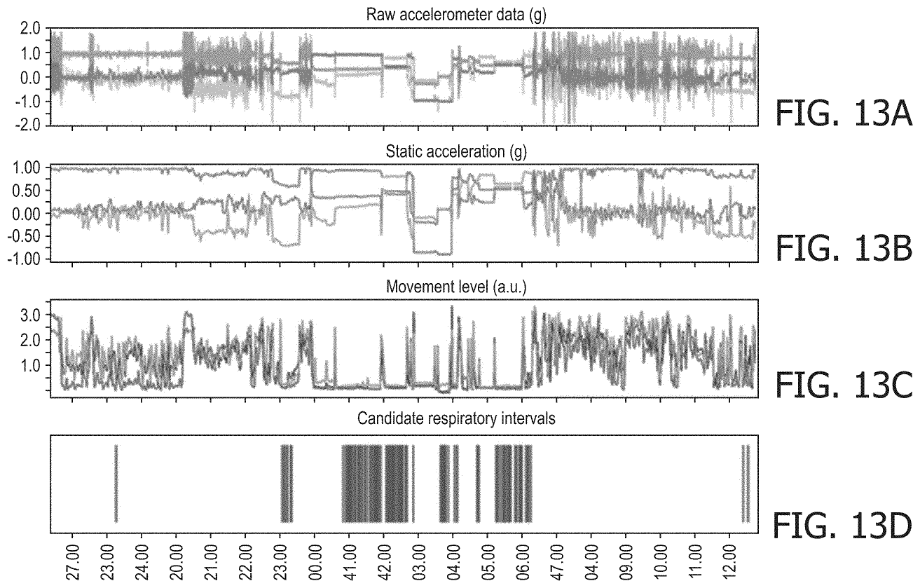

FIGS. 13A to 13D illustrate examples of the operations for identifying groups of samples in acceleration signals; and

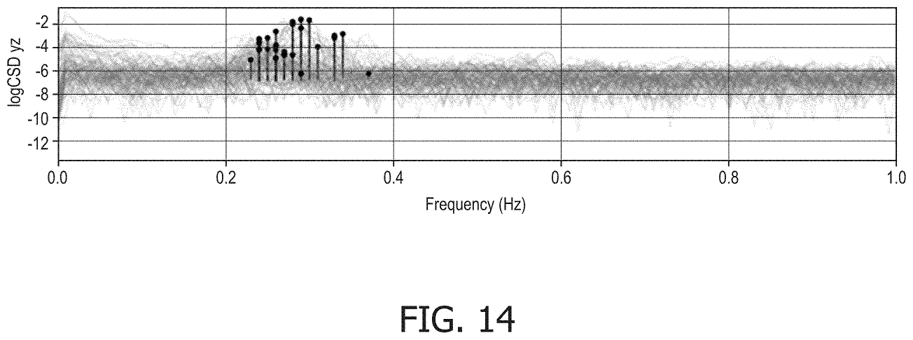

FIG. 14 illustrates examples of multiple cross-spectral power spectral estimates from selected couples of axis for multiple windows.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

Example embodiments describe a system and method for detecting respiratory information based on signals generated by a sensor arrangement that is not fixed to or in continuous contact with the body of a patient. The sensor arrangement may include one or more sensors configured to be included in or on a wearable item (e.g., pendant of a necklace) that comes into intermittent physical contact with at least one body part of the patient throughout the period the sensor arrangement is worn. The intermittent physical contact may be produced by movement of the wearable item relative to the at least one body part, the movement caused by motion or position of the patient.

Because the sensor arrangement is in intermittent contact with one or more portions of the patient's body where meaningful sensor signals may be acquired, the method and device may include features that process the sensor signals to discriminate between periods when the sensor signals constitute only or predominantly noise and periods when the sensor signals include viable respiratory rate information. Once this determination is made, the sensor signals may be processed in order to determine the respiratory rate of the patient. These embodiments may be used by any person, but may be especially useful for patients with asthma, chronic obstructive pulmonary disease, sleep apnea, or any other condition where breathing or respiration rate is a focus of interest.

FIG. 1 illustrates an embodiment of a system 100 for determining respiratory information based on signals generated by a sensor arrangement. In this embodiment, the sensor arrangement 80 is incorporated within a pendant 85 of a necklace 90 worn by a patient whose breathing or respiratory information is to be monitored. While the sensor arrangement is in a pendant-based application in this embodiment, the sensor arrangement may be included in another type of accessory or wearable item that moves and at least intermittently comes into contact with the chest or other body area of a patient. Such intermittent contact may be referred to, for example, as being a loosely fixed application.

Referring to FIG. 1, the system includes a monitoring controller 10, a memory 20, and a storage area 30. In this embodiment, the monitoring controller is connected to the sensor arrangement through a wireless link 95 and interface 50. The wireless link may be, for example, a short-range link including but not limited to a Bluetooth connection or a Wi-Fi connection. When the short-range link is a Bluetooth connection, the monitoring controller 10 may be located in a smartphone or other mobile processing device carried by the patient. In this case, the monitoring controller may be implemented, for example, as an application executed by a processor, processing core, integrated circuit chip, or other form of logic resident in the device.

When the short-range link is a Wi-Fi connection, the monitoring controller 10 may be located in a base station, server, computer, or other processing device connected to a network such as the internet. Alternatively, the monitoring controller may be located at a doctor's office, hospital, a server of a monitoring service, or other medically related facility dedicated to monitoring the condition of the patient. In this case, the signals received from the sensor arrangement 80 through the Wi-Fi connection may be transmitted through the internet to the monitoring controller. To protect the privacy interests of the patient, the network may be implemented as a virtual private network.

In this embodiment, the monitoring controller 10 includes a contact detector 12, a respiratory rate calculator 14, and a processor 16. The contact detector 12 processes the signals received from the sensor arrangement to determine whether the sensor arrangement is in contact with the chest of the patient, at least for a period of time sufficient to acquire meaningful respiratory information from the sensor signals. The respiratory rate calculator 14 is responsive to the contact detector for purposes of identifying, extracting, and/or processing signals from the sensor arrangement and for calculating respiratory rate based on those signals. The processor 16 may generate signals for controlling the contact detector and/or the respiratory rate calculator and for performing additional processing and management functions as described in greater detail herein.

The memory 20 stores instructions which are executed by the monitoring controller for maintaining a connection with the sensor arrangement, determining whether the sensor arrangement is in contact with the chest of the patient (at least for a predetermined period of time), calculating respiratory rate of the patient, and/or communicating that information to the patient directly and/or to a central or medical authority or monitoring service responsible for providing care to the patient. In performing these and other operations, the instructions may embody one or more algorithms for processing the signals received from the sensor arrangement in order to detect contact and calculate respiratory rate, among other operations. Thus, in one embodiment the instructions may be implemented as control programs for application on a device (e.g., smartphone) of the patient.

The storage area 30 may store various types of information. For example, the storage area may store the raw (e.g., unprocessed) signals received from the sensor arrangement over time. These signals may then be sent to the monitoring controller on a continuous, periodic, or other basis for performing the processing described herein. The storage area may also store the processed signals indicative of contact patterns, respiratory rate, and/or other information processed by the monitoring controller. The storage area may also store profile information indicative of the medical history and condition of the patient, communication parameters, processing parameters, and other data and information relevant to the monitoring operations performed by the controller.

The system 100 may also include an output device 40, which, for example, may be a display or other device (with or without a touch screen) capable of indicating results of the processing, providing notifications or warnings based on those results, receiving or inputting commands for controlling the sensor arrangement and/or operational mode and other features of the monitoring controller, receiving input signals for selecting the algorithms and/or other parameters to be monitored, and update software of the monitoring controller, as well as other operations. The operational modes may vary depending on programmed settings indicated by the user or a medical professional. The programmed settings may indicate, for example, a time of day for monitoring the patient, an activity setting for determining when the patient is active or at rest, and/or other settings as will become apparent in accordance with the embodiments herein.

### Sensor Arrangement

The sensor arrangement 80 may include one or more accelerometers that measure movement of the pendant 85, which, in turn, may be used as a basis for detecting contact of the sensor arrangement with the patient and corresponding breathing patterns. However, the sensor signals may not always be reliable for detecting breathing patterns (and thus for determining respiratory rate). In accordance with one or more embodiments, the contact detector 12 and associated algorithms of the monitoring controller may be used to determine one or more sensing periods or time windows when, one, the sensor arrangement is in contact with a body part (e.g., chest) of the patient and, two, when corresponding content of the sensor signals bears meaningful information from which an accurate respiratory rate may be calculated.

For example, when incorporated within or on pendant 85, it is evident that the sensor arrangement will move with the pendant. This movement does not always cause the sensor arrangement to be in contact with the chest of the patient. This may occur at times when the patient bends over or is walking or running, leaning to one side, lying down or sleeping and one side or tossing and turning, or when the patient is performing other forms of activity. When there is little or no contact between the sensor arrangement and the chest of the patient, the acceleration sensor signals may contain only noise or other forms of spurious signals.

To overcome this problem, the contact detector 12 of the monitoring controller processes the sensor signals to detect movement information (e.g., degrees of movement of the patient) along one axis, two axes, or all three directional axes of movement relative to the pendant. The movement along these axes may be used as a basis for determining whether the sensor arrangement is in contact with the chest of the patient, and thus whether the sensor signals are in a sensing period or time window where the signals contain information that may be used to calculate a reliable respiratory rate.

In one embodiment, accelerometers detect movement along three orthogonal axes, two of which are arranged in directions that define a lateral (x-y) plane substantially parallel to the chest of the patient and the third axis arranged in a direction substantially perpendicular to the chest of the patient. However, movement detected along only a subset of these axes is used as a basis for detecting contact and respiratory rate. While accelerometers are used in the sensor arrangements of some embodiments, different types of motion sensor may be used in other embodiments. Examples include magnetometers and gyroscopes. In one embodiment, multiple types of motion sensors may be used in combination (e.g., combinations of magnetometer, gyroscope, and accelerometer) in order to provide a fused motion estimate in accordance with one or more embodiments described herein. That is, in some embodiments, motion estimates may be performed, either alone or with static orientation estimates.

In order to acquire signals indicative of movement (or degrees thereof), the pendant may be worn at a level which aligns the sensor arrangement at an upper thorax or upper portion of the abdomen of the patient. In either of these positions, movement of the body caused by breathing may be at its most significant, thereby making placement of the sensor arrangement suitable at either of these positions.

FIG. 2 illustrates an example of the pendant including the sensor arrangement discussed above. In this example, the sensor signals may be transmitted, for example, from the sensor arrangement to a watch device 210 (e.g., Apple Watch, etc.) through a Bluetooth link. The watch device may include the monitoring controller or the watch device may transmit the sensor signals to a smartphone in the pocket of the patient 220, which smartphone may include the monitoring controller and/or which may transmit the signals (and/or the processed data indicative of respiratory rate) to a remotely located system through a network. In one embodiment, the sensor arrangement may transmit the signals directly to the smartphone or other device of the monitored patient.

The patient may control the operational mode, functional parameters, or on/off state of the sensor arrangement based on signals generated through the watch device and/or smartphone. Thus, in at least one embodiment, the monitoring controller 10 may be implemented as an application on the watch device or smartphone, which connectivity, notification, and processing results may be shown, e.g., the watch device or smartphone may correspond to the output device 50 of the system of FIG. 1. The sensor arrangement may be powered by one or more batteries included, for example, in a housing of the pendant 85 in a manner hidden from view. While FIG. 1 shows the monitoring controller separated from the pendant and sensor arrangement, in one embodiment the monitoring controller may be coupled to the sensor arrangement and implemented in the pendant.

### Respiratory Rate Monitoring

FIG. 3 illustrates an embodiment of a method for determining respiratory information based on signals generated by a sensor arrangement. The method may be performed, for example, by the system of FIG. 1 and the sensor arrangement previously described.

The method determines respiratory rate based on the concept that, during breathing, the thorax moves and expands in a radial fashion. Relying on this motion pattern, for each orientation a patient might be in, the motion and corresponding acceleration measured by the sensor arrangement (e.g., a three-axial accelerometer) will be of higher amplitudes along two dimensions relative to the remaining third dimension. These relative amplitudes may be used as a basis for determining (or selecting) one or more sensing time windows. The signals in these windows are then used for breathing rate calculation.

The determination or selection of the one or more sensing time windows may be performed using at least one of two ranking operations. In one embodiment, both ranking operations may be performed and checked against one another to provide confirmation of the sensing time window(s). The determination or selection of the sensing time window(s) may be performed by contact detector 12 alone or in cooperation with control operations implemented by processor 16. Examples of the two ranking operations are described below, wherein the one or more sensing time windows are considered to be candidate time windows.

Ranking Operation 1: Sensor Amplitude. In the first ranking operation, time windows may be ranked based on the motion levels of their signals, which, for example, may be separately calculated for each axis. Then, one or more time windows may be selected that have low motion levels (e.g., motion level values below a first predetermined value), but ones that are still above a predetermined axis-dependent noise floor (e.g., motion level values above a second predetermined value) for at least two out of three axis. The first predetermined value may be indicative of low patient activity, such as the patient being still, sitting, sleeping, etc. Such a value may be determined based on training data or an initial data set determined for general patients. In one embodiment, the monitoring controller may implement a machine-learning algorithm which learns the motion levels of the patient at low activity periods.

The application of these predetermined values may effectively constitute a filtering process, which, for example, may be performed on a continuous basis (e.g., as long as the monitoring controller or the smartphone, base station, or other host device detects connection to the sensor arrangement), based on a predetermined schedule entered into the monitoring controller by the patient or care professional, or based on an activation signal entered by the user or generated automatically when a link is connected between the sensor arrangement and the monitoring controller.

The accelerometer sensor signals in the selected time windows produced from the ranking operation (e.g., the ones not filtered out) may be considered to be indicative of chest motion signals that may be processed to provide a reliable calculation of the respiratory rate, at least during the corresponding periods corresponding to each of the selected windows. The accelerometer sensor signals corresponding to times or windows that have been filtered out (or otherwise are not included in a selected time window) may be considered noise or spurious signals that may not be depended on to calculate a reliable respiratory rate. These windows may therefore be discarded.

Ranking Operation 2: Sensor Sensitivity. In the second ranking operation, the candidate time windows may be ranked based on accelerometer sensitivity. Accelerometer sensitivity may be determined, for example, based on the average orientation of the sensor arrangement (or pendant). Windows with the highest sensitivity (e.g., once having an average orientation above a predetermined value) along chest expansion directions may be selected as time windows that contain chest motion signals which may be used as a basis for calculating respiratory rate. Other times or windows may be filtered out on the basis that their corresponding signals constitute noise or spurious signals.

Referring now to FIG. 3, the method includes, at 310, receiving signals from the sensor arrangement in the pendant. In one embodiment, the accelerometer signals may indicate movement or acceleration of the sensor arrangement/pendant, but may not necessarily be indicative of movement or acceleration of the body of the patient. In some cases, movement or acceleration of the pendant/sensor arrangement and the body of the patient may be coincident.

As previously indicated, in this embodiment the sensor arrangement includes a tri-axial accelerometer. The sensor signals may be received by the monitoring controller based on one or more signals transmitted to connect and/or activate the tri-axial accelerometer in the pendant. In one embodiment, the tri-axial accelerometer may include an integrated circuit that controls communications with the monitoring controller and operation of the accelerometer based on the one or more signals transmitted to the accelerometer. The control signals may include a user request signal 302 or a periodic trigger signal 304 generated, for example, by an algorithm implemented by the monitoring controller. In one embodiment, the monitoring controller may receive previously collected accelerometer signals 306 stored in a memory of the integrated circuit of the accelerometer or stored in the storage area 30 of the system.

At 315, the contact detector 12 executes an algorithm stored in memory 20 to process the sensor signals to determine whether the patient (or pendant) is moving. The contact detector may perform this operation in association with processor 16. If the pendant (and thus the patient) is determined to be moving (e.g., the amplitude or sensitivity of sensor signals are above one or more predetermined corresponding thresholds), the contact detector may determine that there is no contact (or at least not a sufficient amount of contact to calculate respiratory rate). In this case, the processor of the monitoring controller generates a signal indicating not to perform a respiratory rate calculation, at box 360, because the sensor signals received constitute noise or otherwise are spurious signals and would not yield a reliable respiratory rate measurement. This signal may be transmitted to the respiratory rate calculator 14 or the processor 16 so that the respiratory rate calculation is not performed under these conditions (or if performed discarded).

At 320, if processing of the sensor signals by the processor and/or contact detector indicate that the patient (or pendant) is not moving (e.g., sensor signals below a predetermined threshold), a determination is made by the processor and/or contact sensor as to whether the pendant is being worn by the patient. This determination may involve, for example, detecting that the sensor signals have relatively high amplitudes or sensor sensitivity on all (or at least a subset of) three axes. Such a signal pattern would be generated, for example, when the pendant is lying on a table or nightstand. If operation 320 indicates that the patient is not wearing the pendant or the sensor arrangement is offline (because no signals are being received by the sensors), then the processor does not enable the respiratory rate calculator 14, at operation 360.

At 325, the processor of the monitoring controller determines that the sensor arrangement is online, when operation 320 indicates that the patient is wearing the pendant (e.g., the sensor signals have non-zero amplitudes below a relatively low predetermined threshold or non-zero sensitivity levels). In this case, reconfiguration of a trigger device may be performed, for example, in accordance with the method indicated in FIG. 8.

At 330, after the trigger device has been reconfigured, the respiratory rate calculator 14 alone, or in combination with control of the processor 16, may calculate the respiratory rate of the patient based on the accelerometer sensor signals in one or more time sensing windows determined or selected to have meaningful chest motion signals. The respiratory rate may be calculated in accordance with one or more of the ranking operations previously described for the time sensing window(s) or a single reading may be calculated over a plurality or time sensing windows. The respiratory rate may be calculated based on one or more algorithms stored in memory 120.

FIG. 4 illustrates an embodiment of a method for calculating respiratory rate of the patient as performed by the respiratory rate calculator 14. At 405, the acceleration signals from the sensor arrangement are received and sampled by the processor of the monitoring controller. In one embodiment, the sensor arrangement may be equipped with an analog-to-digital converter (e.g., 12-bit) for obtaining the samples. The samples may then be transmitted to the processor of the monitoring controller.

The sensor signals may be indicative of the movement or acceleration of the sensor arrangement in the pendant (which may or may not be indicative of movement or acceleration of the patient). The sampling rate may be set to provide an accurate identification of the movement of the pendant (and/or patient). The sampling rate may be, for example, on a millisecond level. The accelerometer signals may be received, for example, based on a user request signal, a periodic trigger signal, or on a continuous basis after activation or the sensor and corresponding application go online. In one embodiment, the sensor signals may correspond to previously collected sensor signals stored in an integrated circuit of the sensor arrangement, as previously described.

At 410, a vector magnitude signal is calculated based on the samples of the acceleration sensor signals acquired in operation 405. The vector magnitude signal may be calculated based on Euclidian norm of each one of the samples of the signals detected for two or all three of the spatial axes and their corresponding amplitudes.

At 415, one or more groups of samples of the sensor signals are identified that satisfy one or more criterion. In order to qualify as an identified group, the following two criteria must be satisfied: 1) all samples of sensor signals used to calculate the vector magnitude signal must be below a predetermined threshold and 2) the number of samples in the group must be greater than a predetermined minimum value. The identified groups may be referred to as candidate respiratory intervals (CRIs), which, for example, may correspond to the candidate time windows.

In one embodiment, operation 415 may include detecting the sensor arrangement as being worn by the patient. This may be determined by detecting relatively low motion levels of the sensor arrangement (pendant), e.g., motion levels below a predetermined value along one or more of the three axes of the accelerometer arrangement. Next, the patient may sit down, enter into bed and lie down, or enter into another type of low-motion or sedentary activity. The detection algorithm for identifying one or more CRIs may then be performed. In the case where the patient has gotten into bed, the detection algorithm may be performed when the patient has gone to sleep.

At 420, the samples per axis in each identified group (CRI) may then be subjected to a statistical operation. The statistical operation may be, for example, a type of average of the samples in each group, e.g., median values of the samples in each group may be calculated by one of the contact detector, the respiratory rate calculator, or the processor. These median values may then be stored for subsequent processing. The median values of the samples on a per axis basis (whether for one, two, or all three axes) may provide an indication of the orientation of the pendant (sensor arrangement and/or the patient) in the periods when the samples were collected.

At 425, for each CRI, samples are grouped into overlapping subgroups of consecutive samples of one or more predetermined sizes. The one or more predetermined sizes may correspond to sizes of respective ones of the time sensing windows (CRIs). In one embodiment, the size of each window may be smaller than a minimum CRIs size but longer than a predetermined number of (e.g., ten) expected respiratory cycle durations. In some cases, the size of each window may be many times longer than ten expected respiratory cycle durations or even one or more orders of magnitude longer.

At 430, variance values are calculated for the samples over the overlapping subgroups of each CRI. The variance values may be calculated, for example, based on an average of the squared differences between each sample in the group and a mean value of the samples in the group. This calculation may be done on the accelerometer samples from individual accelerometer axis, or on the vector magnitude signal previously discussed.

The median values calculated in operation 420 may be considered to provide an indication of the orientation of the pendant (and/or patient) and the variance values calculated in operation 430 may be considered to provide an indication of the motion (e.g., motion levels) of the pendant (and/or patient) along each axis or along a combination of axes (e.g., at least two axes).

At 435, a number of the three axes is selected for each time sensing (CRI) window based on the variance values (motion level) and median (orientation) values calculated for each of the axes. The number may include a subset of the three axes, e.g., one axis or at least one combination of two of the three axes. In one case, all three axes may be selected. The decision as to the number and particular axes that are selected for each window may involve the following.

Initially, the number of axes are selected based on the variance values and the median values. The selection may be based on the ranking of the variance of each subgroup, applying one of the two ranking operations previously described, e.g., Ranking operation 1. If a window is selected looking at single axis, or a couple of axes (e.g., selected in each axis such that there are low variance levels, but variance above a predetermined axis-dependent noise floor), then the number of axes to be selected for specific subgroup may correspond to the number of axes for which a certain subgroup was selected. The number of axes may be the number of axes for which a predetermined condition is met, for example, as described in accordance with conditions discussed herein.

Which ones of the three axes are selected per window may be based on the variance values and median values. In one embodiment, the selection may be based on the ranking of the variance of each subgroup, applying one of the two ranking operations previously discussed. Subgroups may be grouped based on their median values (e.g., see FIG. 12B sensor orientation, related to subject posture), and the axis being selected the most times for each subgroup separately may be chosen as the selected axis for the entire cluster, including for subgroups in the cluster for which a different axis would have been selected in first place (e.g. using the variance discussed above).

FIG. 12A illustrates an example of a plot motion levels (log [variance accelerometer axis]) for two arbitrary accelerometer axes. Each point in the plot represents one subgroup of samples, with the horizontal axis in the plot corresponding to the motion level on one specific axis and the vertical axis in the plot corresponding to the motion level on a different axis. In FIG. 12A, points representative of pure sensor noise (e.g., indicating that the sensor arrangement is off the body) are in the lowest left corner of the plot. Black points indicate an arbitrary selection of windows. For these windows, the two axes represented in the plot are the selected ones. The selection of these axes may be based, for instance, on the motion level being in a predetermined range for each axis (e.g., shown in side plots, in each of the histograms the predetermined range emphasized).

FIG. 12B illustrates an example of a plot of median accelerometer signal values for two axes to be used for a selection of windows. Windows with similar median values may further be grouped, for example corresponding to the same body posture, e.g. sitting or lying, as emphasized by dashed lines. For each group, a different couple of axes may be selected.

Returning to FIG. 4, at 440, power spectral estimates are computed based on the samples from the selected axis (power spectrum) or selected combination of axes (cross-spectrum) for each time sensing window. Power spectrum estimates may be obtained by calculating the Fourier transform of a signal. The Fourier transform of the signal can be represented as a 2-dimensional vector, as a complex number, or as magnitude and phase in polar coordinates. A common technique in signal processing is to consider the squared amplitude, or power; in this case the resulting result is referred to as a power spectrum. Power spectrum calculation might include the multiplication of a smoothing window (some arbitrarly preset values) with the original signal before the calculation of the fourier transform, in order to improve spectral resolution and reduce effects of the sequence transformed being of finite length.

At 445, an estimate of respiratory rate is calculated based on each power spectral estimate for each time sensing window. The respiratory rate may be calculated, for example, by determining the frequencies of the peak amplitudes in each power spectral estimate for each time sensing window. The frequencies may be within a certain respiratory rate search range (RR-SR). Alternatively, the estimate of respiratory rate may be determined, as the inverse of the peak-to-peak time in the accelerometer signals is determined as corresponding to the respiratory rate. This approach may be also be taken when the respiratory waveform can be resolved using the modified embodiment of FIG. 8.

At 450, a quality estimate for the peak of each power spectral estimate is computed. The quality estimate for the peak may be calculated using various approaches. One approach involves calculating the signal-to-noise ratio, by dividing the sum of power spectral estimates in RR-SR by the sum of power spectral estimates in a second search range, which, for example, may be the entire acquisition frequency range or sub-selection of the entire acquisition frequency range.

Another approach involves measuring consistency with similar orientations of the sensor arrangement. This approach may involve comparing the peak amplitude value with amplitude values from other peaks from the same axis from one or more different windows and from one or more different identified groups (or CRIs). The difference between the orientation of the current group (from which the current samples have been drawn) and the orientation of each of the one or more different groups may provide an indication of the quality estimate. For example, when the difference is less than a predetermined threshold (e.g., selected to correspond to a desired level of consistency), then the quality estimate may be determined to be of an acceptable level.

Another approach involves determining a difference from the median value. This may involve comparing the peak amplitude value of each power spectral estimate with peak amplitude values of other peaks from a same axis relative to one or more different windows within an identified group (CRI). This difference may provide an indication of the quality estimate.

At 455, the power spectral peak estimates from two or different windows are combined. The combination may be performed, for example, by first averaging spectra from different windows and then detecting the peak based on the average spectrum. The peaks derived from several windows and/or from a different axis, or a combination of two different axes, are then combined into a weighted average. The weight(s) might be determined, for example, based on the quality estimate determined in operation 450 or by some a priori knowledge of sensor characteristics, e.g. higher sensitivity to motion along a specific axis may serve as a basis for assigning a higher weight to peak estimates based on that axis. In one embodiment, the two or more different windows for which the power spectral peak estimates are combined may be a predetermined number of windows that are consecutively or successively arranged or may be ones separated by one or more other windows.

At 460, the power spectral peak estimates from different axes are compared, and a final decision as to the respiratory rate is issued for each group (e.g., for each CRI or time sensing window). For example, the final respiratory rate may be determined based on the motion level, orientation, and peak quality for each window.

In one embodiment, the final respiratory rate may be calculated, for example, by determining the frequencies of the peak amplitudes in each power spectral estimate the subgroup based on the following equations.

$$FR = {\sum }_{i = 1}^{3} {\sum }_{j = 1}^{3} argmax [ FT ( {x}_{i} [ t ] * {w}_{1} [ t ] ) * {FT}^{*} ( {x}_{j} [ t ] * {w}_{j} [ t ] ) ] * {q}_{i , j}$$

### Subject to:

$${\sum }_{i = 1}^{3} {\sum }_{j = 1}^{3} {q}_{i , j} = 1$$

In general, q_{i,j}=q_{i,j}(σᵢ, σⱼ). For instance, q_{i,j}=0 iff σₓᵢ>TH, where xₐ[t] are the accelerometer samples for axis a for each time-point t in the subgroup, FT is the Fourier transform or similar frequency domain transform, wₐ[t] is a windowing function applied for the spectrum calculation, * (apex) indicates complex conjugate, and q_{i,j} is the peak quality. This may be an arbitrary function of the motion level or of some characteristics of the power spectrum as described in accordance with examples herein. Also, σₐ is the motion level for axis a, for instance the variance of xₐ[t].

When i=j, the calculation may be based on the spectrum. Otherwise, the calculation may be based on cross-spectrum, where respiratory rate is based on an inverse of the previously determine frequencies, e.g., RR=1/FR.

Returning to FIG. 3, once the respiratory rate calculation has been performed, then, at 335, the processor 16 may receive this calculation from the respiratory rate calculator 14 and then output the calculated rate(s) in one or more forms. For example, the processor 16 may store the respiratory rates in storage area 30 to create a record of the respiration activity of the patient. This record may be uploaded to a server for analysis by medical professionals charged with monitoring the care of the patient. Also, the calculated rate(s) may be transmitted directly to such a server without being stored in storage area 30. Additionally, or alternatively, the calculated rate(s) may be output on the output device 40 in order to notify the patient. If the rate(s) are determined by the processor 16 to be within one or more ranges that indicate different corresponding severities of deterioration of the patient condition, then an alert may be generated. This may be especially beneficial when the output device is included in a smartphone of the patient, along with the monitoring controller. In some cases, the respiratory rate calculation fails, e.g., because of no suitable CRI identified, or in case of no suitable subgroup of samples identified within a CRI. This may occur, for example, when all windows have variance typical of pure sensor noise, or in case of none detected peaks not having acceptable quality (e.g., signal-to-noise-ratio below a threshold for all peaks). When this happens, process flow may proceed to block 360 indicating that no calculation was able to be provided. In this case, if sensor signals are still being received, the method may be return to initial operation 310 to perform additional attempts at obtaining one or more successful respiratory rate calculations.

FIG. 5 illustrates an embodiment of a method for determining respiratory information based on signals generated by a sensor arrangement. This method embodiment may also be performed, for example, by the system of FIG. 1 and the sensor arrangement previously described. For at least some operations, the embodiment of FIG. 5 may be considered to be a more specific implementation of the method embodiment of FIG. 4.

Initial operations of the method include sampling the accelerometer signals (510), segmenting samples into subgroups (520), and determining motion level and orientation per subgroup per axis (x, y, z). At 530, the motion level in each subgroup may be computed, for example, by calculating variance values of the samples in the subgroup, and the orientation in each subgroup may be computed, for example, by calculating median values of the samples in each subgroup. Operations 510 to 530 may be performed, for example, in a manner similar to operations 405 to 430 in FIG. 4.

At 540, the subgroups are ranked based on motion level as determined in operation 530. The ranking may involve performing one or both of the ranking operations previously described, e.g., ranking by sensor amplitude and ranking by sensor sensitivity. For example, the subgroups may be ranked based on the motion levels of the signals in respective ones of those subgroups, which, for example, may be calculated for each axis. Additionally, or alternatively, the subgroups may be ranked based on accelerometer sensitivity, which, for example, may be determined based on the average orientation of the sensor arrangement (or pendant) for each subgroup.

At 550, subgroups are selected which have motion levels below a preset value and/or above a certain other preset value (e.g., accelerometer axis-dependent noise floor), taking one or more characteristics 580 of the accelerometer sensor into consideration. The preset value may be selected to indicate low motion level of the pendant, which, in turn, may correspond to conditions or periods where the sensor arrangement is in contact with the patient for purposes of obtaining meaningful (e.g., accurate, and not noise) chest motion data to be extracted from the sensor signals.

The sensor characteristics 580 may include, for example, the noise floor of each axis (e.g., which may be used in 550 as a basis to discard subgroups representing only noise), the sensitivity of each axis in the current device operation mode (e.g., which may be used as a basis in 550 to select groups which have motion level in the selected range for the axis with highest sensitivity), and/or the alternative settings for sensitivity (e.g., if better settings are available, then this may be used to discard all windows when the sensitivity of the accelerometer was too low in a specific setting).

At 560, windows are selected which have a motion level above a predetermined noise floor on at least two axes of the tri-axial accelerometer. This operation may also be performed by taking one or more characteristics 580 of the accelerometer sensor into consideration. The predetermined noise floor may correspond to a value indicating that, for example, the pendant is not worn by the patient. The windows may correspond to respective ones of the selected subgroups and may be considered time sensing windows (CRIs) for purposes of calculating respiratory rate. The axis or axes of the accelerometer for which motion levels are above the predetermined noise floor are recorded for use, for example, in operation 615.

At 570, a subset is defined to include the selected subgroups and their corresponding axis/axes. The window corresponding to this subset may be considered to be the time sensing window for calculating respiratory rate. Thus, the samples contained within this window may be considered to correspond to the case where the sensor arrangement in the pendant is in contact with the body of the patient.

FIGS. 6A and 6B illustrate additional operations of the method of FIG. 5. Once the subset of selected groups is determined in operation 570, then the method continues by performing a power spectrum calculation. For example, a power spectrum may be calculated, at 606, based on the samples in the subset of selected groups for each of three axes (x, y, z). Additionally, power spectra may be calculated, at 608, based on the samples in the subset corresponding to one or more of the following two-axis combinations: xy, yz, xz. At this stage, all power spectra may be calculated (e.g., 3 power spectra and 3 power cross-spectra), or only selected ones of the power spectra may be calculated. For example, in one embodiment, a power spectrum (or cross-spectrum) may be calculated only for one axis (or a set of axes) selected for a specific group.

At 610, the peak and peak quality of each calculated power spectrum is determined. This may involve an operation of searching various regions of the frequency domain 612 corresponding to the signals or samples contained in the subset of selected subgroups. The frequency domain may be obtained by applying a predetermined transfer function (e.g., Fourier transform) to the signals in the selected subgroups of the subset. Once a frequency domain representation of the signals is obtained, the frequency domain may be analyzed to determine the peak and peak quality values. The peak quality may be indicative of the prominence and/or sharpness of the peak identified in the power spectrum, and thus may measure of how confident a user can be in the provided frequency (and consequent respiratory rate) estimate.

At 615, a determination is made as to whether the peaks identified in operation 610 are compatible with the axis (or axes) identified in operation 560. This compatibility is determined, for example, by estimating the posture of a subject. Each posture of a subject may be associated with some subset of axes having the highest sensitivity e.g., if based on median value of samples the subject may be deemed to be lying/sleeping. Peaks identified on the accelerometer axis aligned with the medio-lateral body direction may not be compatible with the subject posture. Thus, peaks may be discarded that are found where there should not have been any.

If the peaks are not compatible with the axis/axes identified in operation 560, then, at 618, the window corresponding to the subset may be rejected as being one sufficient to qualify as a time sensing window for respiratory rate calculation, i.e., is a window having samples or sensor signals that cannot reliably be determined correspond to contact of the sensor arrangement with the chest. If the peaks are compatible, the method continues.

At 620, a determination is made as to whether peak quality is compatible with orientation of the sensor arrangement (pendant and/or patient body). This compatibility is determined, for example, by comparing peak quality determined at 610 for the axis identified at 560. When a sufficiently high peak quality is achieved for on the axis selected at 560, then these findings are considered compatible. If the peak quality is not compatible, then the window corresponding to the subset is rejected as being one sufficient to qualify as a time sensing window for respiratory rate calculation. If the peak quality is compatible, the method continues.

At 625, a determination is made as to whether at least two of the peaks have similar frequencies. If at least of the peaks do not have similar frequencies, then the window corresponding to the subset is rejected as being one sufficient to qualify as a time sensing window for respiratory rate calculation. If the peak quality is compatible, the method continues.

Similarity between peaks f1 and f2 may be measured, for example, as percentage (%) difference (f1−f2)/(0.5*(f1+f2))*100 between peak characteristics, which include peak frequency (e.g., x-axis in FIG. 11), peak amplitude (y-axis in FIG. 11), full-width-half-maximum, peak quality explained above, and other characteristics derived from the peak, or the entire spectrum. A peak may be similar to another if their percentage (%) difference is below a certain threshold, e.g., 10%. When they are dissimilar, this may indicate that one of the peaks has not been correctly determined. The assumption is that changes in RR should not be too abrupt, and that respiration representative motion should be measurable on multiple axis.

At 630, a respiratory rate estimate is calculated based on signals or samples along a corresponding axis. Examples for calculating the respiratory rate are discussed in greater detail below.

At 635, these same operations may be performed for other subgroups of sensor signals. A comparison of the results of obtained for the current subgroup (window) may then be compared with the results obtained for one or more other subgroups (windows). These results may include, for example, peak estimates 685 and calculated respiratory rates.

At 640, a determination is made as to whether the peak quality obtained for the current subgroup (e.g., CRI or window) is similar to (e.g., same or within at least a predetermined range or tolerance of) the peak qualities obtained for one or more other subgroups (e.g., CRIs or windows) resulting from the comparison. If the peak qualities are not similar, then the window corresponding to the subset is rejected as being one sufficient to qualify as a time sensing window for respiratory rate calculation. If at least two of the peak qualities are similar, the method continues.

At 645, a determination is made as to whether the respiratory rate calculated for the current subgroup (CRI or window) is similar to (e.g., same or within at least a predetermined range or tolerance of) the respiratory rate obtained for one or more other subgroups (CRIs or windows) resulting from the comparison. If the respiratory rates are not similar, then the window corresponding to the subset is rejected as being one sufficient to qualify as a time sensing window for respiratory rate calculation. If the respiratory rates are similar, the method continues.

At 650, if the respiratory rate and peak quality of the current subgroup is similar to the peak quality and respiratory rate calculated for one or more other (e.g., previous) subgroups, then the current subgroup is added to a collection of subgroups (e.g., CRIs or time sensing windows) to be considered for generating a final decision concerning respiratory rate of the patient.

FIG. 7 illustrates additional operations of the method of FIGS. 5 and 6. Once the collection of subgroups is generated (which, for example, may be a predetermined number of subgroups or the subgroups that have been added to the collection after a period of time), then, at 710, these subgroups are designated as ones that are to be considered for use in calculating a respiratory rate estimate.

At 720, one or more axes are identified as the one(s) having the highest peak qualit(ies) among the subgroups. In one embodiment, only one axis is identified, for example, when the peak quality generated for that axis among the subgroups is substantially greater than (e.g., greater than a predetermined A amount) the peak qualities generated for the other axes. In another embodiment, two of the three axes may be identified as having the highest peak qualities among the subgroups. The subgroups corresponding to these two axes may have considerably higher peak qualities (e.g., greater than the same or another predetermined A amount) than the peak quality for the remaining third axis.

At 730, when two of the axes are selected in operation 720, the power spectra (cross-spectra), previously calculated, are combined for different ones of the subgroups from the collection within the same CRI (e.g., window) and for the same axes, respectively. When one axis is selected in operation 720, the power spectra are combined for different ones of the subgroups from the collection within the same CRI and for the same axis. Power spectra as described in P23 are constituted of a Fourier transform magnitude value at each frequency for which the spectrum is defined. The combination may be achieved, for example, by considering for each frequency value (“frequency bin” or frequency location (e.g. 0 Hz, 0.02 Hz, etc.) values on horizontal axis in FIG. 11)) the maximum/minimum/median/5^{th} percentile/95^{th} percentile of all the spectra to be combined at that frequency location.

At 740, the peak value of the combined spectra generated in operation 730 is determined. Because the combined spectra is in the frequency domain (by virtue of translating corresponding sensor signals by some transfer function H(f)), determining the peak value may involve analyzing the combined spectra to determine the value with the highest value in the frequency domain. Additionally, operation 740 may include calculating the peak quality for the combined spectra. This may be accomplished, for example, in the manner previously described but applied on the combined spectrum, instead of a single spectrum (e.g. amplitude peak/mean spectral amplitude in the rest of frequency spectrum).

At 750, the respiratory rate corresponding to the CRI is calculated and reported. The respiratory rate for the CRI may be calculated, for example, by computing the mean (final value) and standard deviation (confidence intervals for final value) of the individual frequency estimates for the selected subgroups of samples within the CRI, using the frequency estimates obtained. The respiratory rate may be reported, for example, by outputting this information to the output device and/or any of the other notification techniques described herein. In addition, the quality may be reported with the respiratory rate. The quality referred to above may correspond to the quality of an estimate for a CRI, which may be determined, for example, by a summary (one or more) statistics of the peak quality of the individual peaks identified in the subgroups selected to provide the frequency estimate within the CRI. Such quality could be indicated, for instance, by the minimum or maximum peak quality for the selected peaks or the variability (e.g. standard deviation) of such peak quality with CRI. In practical terms, the quality indicator may provide information to an end-user about the reliability of the provided estimate over a period of time including multiple RR estimates. Often the user/healthcare professional will be interested in RR over a period (CRI) rather than in specific moment (subgroups within CRI). When the quality would be high, it would indicate the individual peaks being of high quality and therefore the final result being reliable.

FIG. 8 illustrates an embodiment of a method for configuring the sensor arrangement, and specifically for controlling the sensitivity of the sensor arrangement used in connection with one or more embodiments described herein.

The sensitivity of the accelerometer sensor arrangement may be adjusted for a variety of reasons and in a variety of circumstances. For example, accelerometer sensitivity may be adjusted to be at a minimal or other predetermined sensitivity when little or no movement is detected for relatively long interval of time, e.g., indicating sedentary/lying behavior. The relatively long interval of time may correspond to a predetermined amount of time incorporated within the instructions controlling the processor of the monitoring controller. When more significant movement is detected or when one or more triggers occur, the sensitivity of the sensor arrangement may be restored or changed to another sensitivity level (e.g., high), for example, based on the prevailing circumstances as determined by programming. These circumstances may include, but are not limited to, when motion is detected. The sensitivity may be adjusted to high in these circumstances in order to minimize the interference and effects caused by anomalies, e.g., patient falling or bumping into objects. If a commercially available accelerometer is used, such an accelerometer may have selectable measurement ranges of ±2 g, ±4 g, and ±8 g. Therefore, in such a case sensitivity may be adjusted to within various g ranges.

Referring to FIG. 8, the method includes, at 810, detecting a first trigger from the sensor arrangement. The first trigger (Trigger1) may be, for example, may be a patient getting into bed for the night. This trigger may be determined to exist, for example, based on relatively low movement detected from the sensor signals and the time of day, which, for example, may match a pattern of the normal bedtime of the patient. This trigger and the pattern may be stored, for example, in the storage area of the monitoring controller and the detection operation may be performed based on an algorithm stored in memory 120.

At 820, the configuration of the sensor arrangement, as incorporated within the pendant of the patient, is determined. This operation may be performed when the sensor arrangement is integrated into a single tri-axial accelerometer or when the sensor arrangement includes multiple accelerometers, each detecting movement along different axes. The configuration of the sensor arrangement may correspond to, for example, a currently selected measurement range for one or more accelerometers, e.g., one of the ±2 g, ±4 g, and ±8 g mentioned above.

In one embodiment, the optimal configuration for respiratory frequency estimation may be the one with the lowest measurement range, which gives the highest sensitivity. In one example, sensitivity may correspond to the measurement range/2^{N}, where N is the number of bits which are typically fixed. However, the configuration considered to be optimal for respiratory rate (RR) detection for one use case may not be optimal for other use cases, e.g., for fall detection where a higher measurement range may be desirable, or for gait monitoring where the largest measurement range may be preferred in order to correctly measure whole body accelerations. Hence, in accordance with one or more embodiments, the measurement range may be changed for different scenarios.

Information indicative of the configuration of the sensor arrangement is stored in the storage area 130 or another location (Block 870).

At 830, when it is determined in operation 820 that the sensor arrangement has a predetermined configuration, then the processor of the monitoring controller may adjust the sensitivity of the sensor arrangement to a lowest sensitivity. For analog output sensors, sensitivity may be proportional to supply voltage. Thus, doubling the supply voltage may, for example, double the sensitivity. Higher sensitivity may therefore be obtained for short periods of time at the expense of power. Some sensor(s) in the sensor arrangement may be include controls that allow for adjustment of sensitivity. In these cases, the controls may therefore be adjusted to achieve a desired sensitivity.

At 840, in the case where multiple accelerometers are used, each detecting movement along a different axis, the processor of the monitoring controller may transmit signals to selective control the mode of operation of the sensors. For example, one or more of the transmitted signals may selectively turn on one or more of the accelerometers along axes determined to be receiving chest motion data indicative of respiratory rate. One or more other ones of the transmitted signals may selectively deactivate, turn off, or place in a low-power mode one or more of the accelerometers that are configured to measure movement along an axis or axes that are not determined to be of use for detecting chest motion signals, given the current configuration of the sensor arrangement.

At 850, a second trigger (Trigger2) is detected. The second trigger may correspond, for example, to detection of the patient getting out of bed. This detection operation may be performed, for example, in the manner described in EP-2741669-B1, the contents of which are incorporated herein by reference, for all purposes.

At 860, with the patient now out of bed and active, the sensor configuration indicated in operation 370 may no longer be applicable for purposes of contact detection and respiratory rate calculation. In this case, the stored settings for the sensor arrangement may be restored, for example, from the present sensor configuration indicated in operation 870 to a default configuration or one or more other predetermined configurations for purposes of sensing future accelerometer signals.

FIGS. 9A to 9C illustrate examples of joint distributions of motion levels between different axes of the sensor arrangement. FIG. 9A illustrates a distribution of motion levels along a first two-axis combination, namely the z axis (plotted vertically) and the x axis (plotted horizontally). FIG. 9B illustrates a distribution of motion levels along a second two-axis combination, namely the z axis (plotted vertically) and the y axis (plotted horizontally). FIG. 9C illustrates a distribution of motion levels along a third two-axis combination, namely the y axis (plotted vertically) and the x axis (plotted horizontally). The third two-axis combination plots motion in a plane parallel to the chest of the patient being monitored.

In the plot distributions of FIGS. 9A to 9C, the vertical and horizontal values are expressed as a logarithmic function of acceleration variance along respective ones of the axes, e.g., log 10(varZ) indicates logarithmic values of variance along the z axis, e.g., perpendicular to the chest of the patient being monitored. Each plot distribution includes a plurality of squares. Each square may correspond to a different window having a color or shading indicating a corresponding motion level of the patient. The shading of the squares correlates with the motion detected, e.g., greater levels of detected motion correspond to brighter or lighter shading. Each plot also includes two circles, each indicating areas where the motion level is above a certain threshold but below another preset value for each of the two represented axis. Circles are stretched as each axis could have different threshold values. FIG. 10 illustrates an example of a graph of accelerometer signals obtained in accordance with one or more of the embodiments described herein. In this graph, time (in hours) is plotted along the horizontal axis and normalized amplitude of the accelerometer signals is plotted along the vertical axis. Also, the noise floor for the accelerometer signals is a normalized amplitude of −2.0. The accelerometer signals are plotted in normalized form (e.g., normalized by g=gravity) for continuously for three axes over the time axis. Accelerometer signals along the y axis are plotted in blue color and correspond to curve Y, which may represent a median of the y-axis accelerometer signals. Accelerometer signals along the x axis are plotted in red color and correspond to curve X, which may represent a median of the x-axis accelerometer signals. Accelerometer signals along the z axis are plotted in green color and correspond to curve Z, which may represent a median of the z-axis accelerometer signals.

In FIG. 10, the accelerometer signals for each axis occur in a plurality of time windows, which are ranked on distance over the time axis. For example, windows are ranked on distance from the bottom left corner of the plot (where the signals indicate more motion than noise) and on distance from the x=y line (e.g., indicative of motion level difference across axes). The search region for windows within box 1050 may be identified as the CRI, which corresponds to a time when the contact sensor detects contact between the sensor arrangement in the pendant and the chest of a monitored patient. The accelerometer signal samples 1010 within this time sensing window 1050 are generated based on a combination of the accelerometer signals taken along the x axis and the y axis during the window time period. These signals may be used by the respiratory rate calculator to calculate a respiratory rate for the patient during this time window when the probability of contact detected by the contact sensor is significant.

FIG. 11 illustrates a graph including examples of multiple cross-spectral estimates calculated for multiple windows within one or more of the same CRIs. In this graph, frequency is plotted horizontally and normalized amplitude is plotted vertically. The gray line corresponds to individual cross-spectra from individual windows and the three black curves 1110, 1120, and 1130 correspond to respective combinations of axes (e.g., xy, xz, yz) given by maximum, median, and mean value for the cross-spectrum for the same frequency bin across different cross-spectra within a CRI.

FIGS. 13A to 13D illustrate examples of operations for identifying groups of samples in acceleration signals where 1) for all samples the motion level (e.g., variance values of the group of samples) is below a predetermined threshold and 2) the number of samples in the group in greater than a minimum value. These features may correspond, for example, to operation 415 in the embodiments previously discussed. FIG. 13A illustrates examples of raw accelerometer signals. FIG. 13B illustrates examples of median values of each group of samples per axis. FIG. 13C illustrates examples of motion level of each group of samples per axis. And, FIG. 13D illustrates examples of identified groups of samples of acceleration samples (e.g., candidate respiratory levels). The examples in this features also correspond to the stored median values of each group of samples per axis (e.g., orientation of the sensor arrangement in the period when the samples are collected).

FIG. 14 illustrates examples of multiple cross-spectral power spectral estimates from selected couples of axis for multiple windows, as generated in accordance with one or more operations of the embodiments previously described. Also illustrated are one or more quality estimates for the peak, e.g., examples of quality estimates are shown where the value of the peaks correspond to the black points and the prominence is given with respect to a predefined baseline value, e.g. median power in near bands.

### ADDITIONAL FEATURES

In one embodiment, the sensor arrangement may be connected to one or more other sensor arrangements, for example, via wireless transmission, in order to receive updates on internal parameters or in order to dispose of or control additional sensors other than an accelerometer.

In one embodiment, the suitability of a CRI for respiratory frequency estimation may be confirmed using another sensor (e.g., an environmental sensor), which, for example, may be used as a basis for determining whether the patient is lying in bed or is involved in other low-motion activity. An example of such an environmental sensor include a pressure sensor in the bed of the patient. Another type of sensor that may be used for confirmation is a non-contact sensor, e.g. video-based chest-plethysmography, which might be available only intermittently.

In one embodiment, the search range (SR) for the respiratory rate may be determined using an additional, possibly intermittently acquired respiratory rate estimate. The additional estimate for the respiratory rate may be based on measurements that, for example, are more obtrusive or ones corresponding to an indirect estimation via heart rate variability/other vital signs.

One or more embodiments described herein therefore represent a significant improvement in the art of patient monitoring. For example, in accordance with one or more embodiments, the system and method may be implemented without requiring the patient to go to a hospital or other clinical setting. This makes implementation of these embodiments more convenient for the patient and avoids the delays associated with the use of existing respiratory rate monitors.

Additionally, the wearable support of the sensor arrangement may be administered by the patient and operated without any special knowledge or training. After an optional preliminary subscription or initialization procedure, the patient simply puts on the wearable support containing the sensor arrangement and respiratory rate is automatically computed in accordance with one or more algorithms of the disclosed embodiments.

Also, the wearable support and sensor arrangement may not be fixed to the patient for purposes of obtaining accurate readings. This allows the patient to perform activities that would normally be performed at home, work, and/or in other non-clinical settings, while wearing the device for obtaining respiratory rate readings. These embodiments, therefore, do not in any way infringe on the patient's normal lifestyle.

Also, because the system and method do not have to be implemented or otherwise used in a clinical setting, but rather may be continuously operative as long as the patient is wearing the device (e.g., all day and night), the time window for monitoring respiratory rate is not limited in any way, as is the case with existing respiratory rate monitors.

Also, in one or more embodiments, the wearable support may be in the form of jewelry, a clothing accessory, or another type of wearable item that conceals the sensor arrangement or makes the arrangement inconspicuous to observers. As a result, these embodiments may preserve the privacy interests of the patient wearing the device.

The methods, processes, and/or operations described herein may be performed by code or instructions to be executed by a computer, processor, controller, or other signal processing device. The code or instructions may be stored in a non-transitory computer-readable medium in accordance with one or more embodiments. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein.

The controllers, processors, detectors, calculators, filters, and other information generating, processing, and calculating features of the embodiments disclosed herein may be implemented in logic which, for example, may include hardware, software, or both. When implemented at least partially in hardware, the controllers, processors, detectors, calculators, filters, and other information generating, processing, and calculating features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the controllers, processors, detectors, calculators, filters, and other information generating, processing, and calculating features may include, for example, a memory or other storage device for storing code or instructions to be executed, for example, by a computer, processor, microprocessor, controller, or other signal processing device. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods herein.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

Although the various exemplary embodiments have been described in detail with particular reference to certain exemplary aspects thereof, it should be understood that the invention is capable of other example embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

We claim:

1. A method for monitoring a patient, comprising:
receiving sensor signals from a sensor arrangement;
extracting movement information from the sensor signals;
determining a sensing period between the sensor arrangement and a body part of a patient based on the movement information; and
determining a respiratory rate of the patient based on the sensor signals occurring during the sensing period, wherein the sensor signals are received from a sensor arrangement incorporated on or within a wearable item that moves relative to the body part of the patient, the sensor arrangement in intermittent patterns of contact and non-contact with patient as a result of movement of the wearable item.

2. The method of claim 1, wherein the wearable item is a pendant on a necklace.

3. The method of claim 1, wherein the sensing period includes a period of contact between the sensor arrangement and the body part of the patient.

4. The method of claim 3, wherein determining the sensing period includes:
determining one or more periods of non-contact between the sensor arrangement and the body part of the patient, and
excluding the one or more periods of non-contact to determine a period of contact between the sensor arrangement and the body part of the patient, the period of contact corresponding to the sensing period.

5. The method of claim 1, wherein the movement information indicates movement of the wearable item along a subset of three directional axes.

6. The method of claim 5, wherein the subset includes:
one axis, and excluding the remaining two axes, of the three directional axes, or
a combination of two of the three directional axes.

7. The method of claim 5, further comprising:
combining the sensor signals generated along the combination of two of the three directional axes to generate the movement information.

8. The method of claim 5, wherein determining the period of contact includes:
determining at least one time window where the movement information indicates that movement of the wearable item along the subset of three directional axes is below at least a first predetermined value.

9. The method of claim 8, wherein the first predetermined value is indicative of a sitting state, a lying down state, standing still, or a sleep state.

10. The method of claim 8, wherein determining the at least one time window includes:
identifying a plurality of candidate time windows,
ranking the candidate time windows based on at least one parameter, and
selecting the at least one time window from the plurality of candidate time windows,
wherein the at least one parameter corresponds to at least one parameter of the sensor signals in each of the plurality of candidate time windows and wherein unselected ones of the candidate time windows are discarded as containing noise or spurious signals.

11. The method of claim 10, wherein the at least one parameter of the sensor signals is based on amplitudes of the sensor signals in the plurality of candidate windows.

12. The method of claim 10, wherein the at least one parameter of the sensor signals is based on sensitivity of the sensor arrangement.

13. The method of claim 10, wherein the at least one parameter of the sensor signals is based on a median value of the sensor signals in the plurality of candidate time windows.

14. The method of claim 1, further comprising:
generating median values based on amplitudes of the sensor signals during one or more candidate respiratory intervals corresponding to the sensing period, the median values generated for at least a subset of three directional axes and indicative of one or more corresponding orientations of the wearable item;
generating variance values for the sensor signals during the one or more candidate respiratory intervals corresponding to the sensing period, the variance values, the variance values generated for at least the subset of the three directional axes and indicative of one or more corresponding motion levels of the wearable item; and
determining the period of contact between the sensor arrangement and the body part of a patient based on one or more of the median values and one or more of the variance values.

15. The method of claim 14, wherein determining the respiratory rate includes:
generating power spectral and cross-spectral estimates based on the sensor signals in the sensing period; and
calculating the respiratory rate based on the power spectral estimates.

16. A monitor, comprising:
a memory configured to store instructions; and
a processor configured to execute the instructions to generating information for a patient to be monitored, the processor including:
(a) a contact detector configured to receive sensor signals from a sensor arrangement, extract movement information from the sensor signals, and determine a sensing period between the sensor arrangement and a body part of a patient based on the movement information, and
(b) a respiratory rate calculator configured to determine a respiratory rate of the patient based on the sensor signals occurring during the sensing period, wherein the sensor signals are received from a sensor arrangement incorporated on or within a wearable item that moves relative to the body part of the patient, the sensor arrangement in intermittent patterns of contact and non-contact with patient as a result of movement of the wearable item.

17. The monitor of claim 16, wherein the sensing period includes a period of contact between the sensor arrangement and the body part of the patient.

18. The monitor of claim 16, wherein determining the sensing period includes:
determining one or more periods of non-contact between the sensor arrangement and the body part of the patient, and
excluding the one or more periods of non-contact to determine a period of contact between the sensor arrangement and the body part of the patient, the period of contact corresponding to the sensing period.

19. The monitor of claim 16, wherein the movement information indicates movement of the wearable item along a subset of three directional axes.

20. The monitor claim 19, wherein the subset includes:
one axis, and excluding the remaining two axes, of the three directional axes, or
a combination of two of the three directional axes.

21. The monitor of claim 19, wherein the contact detector is configured to combine the sensor signals generated along the combination of two of the three directional axes to generate the movement information.

22. The monitor of claim 19, wherein the contact detector is to determine the sensing period by determining at least one time window where the movement information indicates that movement of the wearable item along the subset of three directional axes is below at least a first predetermined value.

23. The monitor of claim 22, wherein the first predetermined value is indicative of a sitting state, a lying down state, standing still, or a sleep state.

24. The monitor of claim 22, wherein the contact detector determines the at least one time window by:
identifying a plurality of candidate time windows,
ranking the candidate time windows based on at least one parameter, and
selecting the at least one time window from the plurality of candidate time windows,
wherein the at least one parameter corresponds to at least one parameter of the sensor signals in each of the plurality of candidate time windows and wherein unselected ones of the candidate time windows are discarded as containing noise or spurious signals.

25. The monitor of claim 24, wherein the at least one parameter of the sensor signals is based on:
amplitudes of the sensor signals in the plurality of candidate windows,
sensitivity of the sensor arrangement, or
both.
